(19) United States
(12) Patent Application Publication
(10) Pub. No.: US 2018/0259530 A1
(43) Pub. Date: Sep. 13, 2018

(54) Method and kit for testing chronic myelogenous leukemia (CML), method for isolating TKI-resistant CML cells, and agent for reducing TKI-resistance of CML and method for screening the same

(71) Applicant: The University of Tokyo, Tokyo (JP)

(72) Inventors: Masashi Miyauchi, Tokyo (JP); Mineo Kurokawa, Tokyo (JP)

(73) Assignee: The University of Tokyo, Tokyo (JP)

(21) Appl. No.: 15/895,603

(22) Filed: Feb. 13, 2018

(30) Foreign Application Priority Data
Feb. 15, 2017 (JP) 2017-026287
Oct. 30, 2017 (JP) 2017-209442

(51) Int. Cl. G01N 33/574

(52) U.S. Cl. CPC G01N 33/57426; G01N 2800/54; G01N 2800/52; G01N 2800/56

(57) ABSTRACT

One or more embodiments of the invention relate to a testing method and kit for testing chronic myelogenous leukemia (CML). One or more embodiments of the invention also relate to a method for isolating tyrosine kinase inhibitor (TKI)-resistant CML cells. One or more embodiments of the invention also relate to an agent for reducing TKI resistance in CML, and to a screening method for the same.

### TECHNICAL FIELD

One or more embodiment of the present invention relates to a method and kit for testing chronic myelogenous leukemia (CML). One or more embodiment of the present invention also relates to a method for isolating tyrosine kinase inhibitor (TKI)-resistant CML cells. One or more embodiment of the present invention also relates to an agent for reducing TKI-resistance of CML and method for screening the same.

### BACKGROUND

### Chronic Myelogenous Leukemia

CML is a myeloproliferative neoplasm originated in hematopoietic stem cells (HSC), by a BCR-ABL chimeric oncogene arose from t(9;22)(q34;q11) chromosomal translocation (NPL 1). The BCR-ABL chimeric oncogene causes increased proliferation of myeloid cells, that have differentiation potency. When untreated, chronic phase CML (CML in chronic phase: CML-CP) progresses to the accelerated phase, and finally to the blast stage.

Tyrosine kinase inhibitors (TKI) such as imatinib that target BCR-ABL protein improve survival in chronic myelogenous leukemia. Continuation of TKI treatment can inhibit progression from the chronic phase to the accelerated phase and blast phase.

TKI treatment is an effective means for CML, but is problematic due to the side-effects (myelosuppression, nausea, vomiting and diarrhea., etc.) associated with long-term administration of TKI, and the high medical expense. It is therefore desirable to provide a method for classifying patients that need to continue TKI treatment and patients that may discontinue treatment.

Methods for aiding diagnosis and methods for monitoring curative effects for CML are known, that involve measuring expression levels of BCR-ABL (NPL 2). Such methods allow aid in diagnosis and monitoring of therapeutic effect for CML, based on the ratio of BCR-ABL mRNA and ABL mRNA in the RNA extracted from target peripheral blood leukocytes.

However, even if expression of the BCR-ABL gene is not detected using the method, for example, described in NPL 2, and even in patients for whom continuation of TKI treatment is deemed unnecessary, CML sometimes relapses after the TKI treatment has been terminated. This is because TKI-resistant CML cells may be present in those patients in amounts that cannot be detected by the method.

### ADAM8

The ADAM (A Disintegrin And Metalloprotease) family of proteins are proteins having a common domain structure comprising a pro-domain, a metalloprotease domain, a disintegrin-like domain, a cysteine-rich domain, a transmembrane domain and a cytoplasmic domain. These proteins have two functions, that of proteolysis and that of intercellular binding. One of them, ADAM8, is a molecule isolated from macrophage cell lines, and EST analysis has shown that it is expressed in hematopoietic tissue in vertebrates including humans and mice, and in renal and hematopoietic tissue in zebrafish, suggesting that it contributes to the development of blood and circulatory organs. It is known that suppressing expression of ADAM8 inhibits blood circulation and leads to thrombus formation (PTL 1).

ADAM8 is known to be expressed in the malignant epithelia of pulmonary squamous carcinoma, and in pulmonary adenocarcinoma, colon cancer, prostate cancer, breast cancer and inflammatory tissue (PTL 2). In addition, it is known that measurement of ADAM8 levels in biological samples from a subject allows assessment of the development of non-small-cell lung carcinoma (NSCLC) in the subject (PTL 3).

### CITATION LIST

### Patent Literature

[PTL 1] Japanese Unexamined Patent Publication (Kokai) No. 2010-275216
[PTL 2] Japanese Unexamined Patent Publication (Kohyo) No. 2001-513995
[PTL 3] Japanese Unexamined Patent Publication (Kohyo) No. 2007-530921

### Non-Patent Literature

[NPL 1] Nguyen, L. V. et al., Nat. Rev. Cancer 12, 133-43 (2012)
[NPL 2] Nakamae, H. et al., Int J Hematol., 102, 304-311, 2015

### SUMMARY

One or more embodiments of the invention include the following.

[1]

A method for testing chronic myelogenous leukemia, comprising a step of detecting expression of the ADAM8 gene in blood cells in a sample obtained from a subject.

[2]

The method according to [1], wherein the subject is a subject undergoing treatment with a tyrosine kinase inhibitor.

[3]

The method according to [1] or [2], wherein the subject is a subject that has reached MMR (major molecular response) or MUL (molecular undetectable leukemia) by treatment using a tyrosine kinase inhibitor.

[4]

The method according to any one of [1] to [3], which is a method that aids in classification of a patient in need of treatment for chronic myelogenous leukemia.

[5]

The method according to any one of [1] to [3], which is a method that aids in predicting relapse of chronic myelogenous leukemia.

[6]

The method according to any one of [2] to [5], wherein the tyrosine kinase inhibitor is selected from the group consisting of imatinib, gefitinib, erlotinib, osimertinib, afatinib, dasatinib, bosutinib, vandetanib, sunitinib, axitinib, pazopanib, lenvatinib, lapatinib, nintedanib, nilotinib, ibrutinib and ponatinib.

[7]

The method according to any one of [1] to [6], wherein the sample is peripheral blood or bone marrow fluid.

[8]

The method according to any one of [1] to [7], wherein the blood cells are CD34-positive cells.

[9]

The method according to any one of [1] to [8], wherein detecting expression of the ADAM8 gene includes measurement of the number of cells expressing the ADAM8 gene.

[10]

The method according to any one of [1] to [8], wherein detecting expression of the ADAM8 gene includes measurement of the amount of mRNA transcribed from the ADAM8 gene.

[11]

The method according to any one of [1] to [8], wherein detecting expression of the ADAM8 gene includes measurement of the amount of protein encoded by the ADAM8 gene.

[12]

A method for isolating chronic myelogenous leukemia cells having resistance to a tyrosine kinase inhibitor from blood cells in a sample obtained from a subject, the method comprising steps of:

(1) detecting expression of the ADAM8 gene in blood cells in a sample; and

(2) isolating chronic myelogenous leukemia cells having resistance to the tyrosine kinase inhibitor, based on the detection results of step (1).

[13]

A kit for testing chronic myelogenous leukemia, comprising a detection reagent for mRNA transcribed from the ADAM8 gene or a detection reagent for a protein encoded by the ADAM8 gene.

[14]

A composition for lowering tyrosine kinase inhibitor resistance in chronic myelogenous leukemia, comprising an ADAM8 gene expression inhibitor or an ADAM8 activity inhibitor.

[15]

The composition according to [14], wherein the ADAM8 gene expression inhibitor is selected from the group consisting of antisense oligonucleotides, RNAi-inducible nucleic acids, microRNA (miRNA), ribozymes, genome editing nucleic acids and their expression vectors, low molecular weight organic substances, aptamers, antibodies, antibody fragments, and combinations thereof.

[16]

The composition according to [14], wherein the ADAM8 activity inhibitor is selected from the group consisting of low molecular weight organic substances, aptamers, antibodies, antibody fragments, and combinations thereof.

[17]

A composition for treatment of chronic myelogenous leukemia comprising a tyrosine kinase inhibitor, wherein the composition is administered to a subject in combination with an ADAM8 gene expression inhibitor or ADAM8 activity inhibitor.

[18]

A method for screening an agent for lowering tyrosine kinase inhibitor resistance in chronic myelogenous leukemia, wherein ADAM8 gene expression inhibition or ADAM8 activity inhibition is used as the marker.

[19]

The method according to [18], comprising steps of:

contacting ADAM8 gene-expressing cells with a test substance;

measuring the expression level of the ADAM8 gene in the cells;

comparing the expression level of the ADAM8 gene measured in the previous step with a control expression level, the control expression level being the expression level of the ADAM8 gene in ADAM8 gene-expressing cells that have not been contacted with the test substance; and

selecting a test substance that lowers the expression level of the ADAM8 gene in comparison with the control expression level.

[20]

The method according to [18], comprising steps of:

contacting ADAM8 gene-expressing cells with a test substance;

measuring ADAM8 activity in the cells;

comparing the ADAM8 activity measured in the previous step with a control activity, the control activity being the ADAM8 activity in ADAM8 gene-expressing cells that have not been contacted with the test substance; and

selecting a test substance that lowers the activity of the ADAM8 gene in comparison with the control activity.

[21]

The method according to [18], comprising steps of:

contacting ADAM8 with a test substance;

measuring the ADAM8 activity;

comparing the ADAM8 activity measured in the previous step with a control activity, the control activity being the activity of ADAM 8 that have not been contacted with the test substance; and

selecting a test substance that lowers the ADAM8 activity in comparison with the control activity.

[22]

A method for lowering tyrosine kinase inhibitor resistance in chronic myelogenous leukemia, comprising a step of administering an ADAM8 gene expression inhibitor or an ADAM8 activity inhibitor to a subject suffering from tyrosine kinase inhibitor-resistant chronic myelogenous leukemia.

[23]

A method for treatment of chronic myelogenous leukemia, comprising a step of administering a tyrosine kinase inhibitor to a subject in need of treatment for chronic myelogenous leukemia, in combination with an ADAM8 gene expression inhibitor or an ADAM8 activity inhibitor.

[24]

A tyrosine kinase inhibitor for treatment of chronic myelogenous leukemia, which is administered to a subject in combination with an ADAM8 gene expression inhibitor or ADAM8 activity inhibitor.

[25]

Use of a tyrosine kinase inhibitor in the manufacture of a composition for treatment of chronic myelogenous leukemia, wherein the composition is administered to a subject in combination with an ADAM8 gene expression inhibitor or ADAM8 activity inhibitor.

### BRIEF DESCRIPTION OF THE DRAWINGS

FIG. 1 shows RT-PCR analysis results for endogenous stem cell gene and BCR-ABL expression in iPSC taken from a CML-CP patient.

FIG. 2 shows the experiment scheme for Example 1(1).

FIG. 3 shows the results for a cell proliferation assay in the absence of imatinib.

FIG. 4 shows the results for a cell proliferation assay in the presence of 2.5 μM imatinib.

FIG. 5 shows the results of an apoptosis assay.

FIG. 6 shows the evaluation results for expression levels of IK1RL1 and MEIS1 in pre-HPCs and DCs, in the presence of or in the absence of imatinib.

FIG. 7 shows the results of evaluating relative mRNA expression levels of ADAM8 after imatinib treatment, using real-time quantitative RT-PCR.

FIG. 8 shows the results of a cell viability assay of ADAM8-positive CML-pre-HPC cells and ADAM8-negative CML-pre-HPC cells.

FIG. 9 shows separation of a CD34⁺ADAM8⁺ cell fraction and a CD34⁺ADAM8⁻ cell fraction by FACS.

FIG. 10 shows the results of frequency measurement of ADAM8+ cells in CD34+ cell fractions taken from a CML-CP patient, an NHL patient (normal control) and another malignant blood disease patient.

FIG. 11 shows the results of a cell viability assay using CD34+ADAM8+ cells and CD34+ADAM8− cells.

FIG. 12 shows the results of an apoptosis assay using CD34+ADAM8+ cells and CD34+ADAM8− cells.

FIG. 13 shows the results of frequency measurement of ADAM8+ cells in CD34+CD38+ cell fractions and CD34+CD38− cell fractions taken from a CML-CP patient, an NHL patient (normal control) and another malignant blood disease patient.

FIG. 14 shows separation of CD34+CD38+ADAM8+ cell fractions and CD34+ADAM8− cell fractions by FACS.

FIG. 15 shows the results of a cell viability assay using 8CD34+CD38+ADAM8+ cells and CD34+CD38+ADAM8− cells.

FIG. 16 shows the results of an apoptosis assay using CD34+CD38+ADAM8+ cells and CD34+CD38+ADAM8− cells.

FIG. 17 shows the frequency of BCR-ABL-positive cells in a sample from a patient that had reached MMR and MUL by TKI treatment.

FIG. 18 shows the results of evaluating the efficacy of shRNA that inhibits expression of the ADAM8 gene.

FIG. 19 shows the results of a cell viability assay in the absence of and in the presence of imatinib, after treatment of ADAM8 gene-expressing cells with shRNA that inhibits expression of the ADAM8 gene.

FIG. 20 shows the results of a cell viability assay when ADAM8 gene-expressing cells were treated with GM6001.

FIG. 21 shows the results of a cell viability assay in the absence of and in the presence of imatinib, after treatment of CML-CP9-derived ADAM8 gene-expressing cells with GM6001.

FIG. 22 shows the results of a cell viability assay in the absence of and in the presence of imatinib, after treatment of CML-CP23-derived ADAM8 gene-expressing cells with GM6001.

### DESCRIPTION OF EMBODIMENTS

The present inventors have found that ADAM8 can be used as a biomarker for TKI-resistant CML cells remaining after TKI treatment. The present inventors have further found that TKI resistance in CML cells can be reduced by inhibiting ADAM8 gene expression or ADAM8 activity.

### <Testing Method of the Invention>

According to the first aspect, the invention relates to a method for testing chronic myelogenous leukemia, comprising a step of detecting expression of the ADAM8 gene in blood cells in a sample obtained from a subject. This will hereunder also be referred to as “testing method of the invention”.

As used herein, “testing” means detection of ADAM8 gene expression in a sample obtained from a subject, in order to obtain information necessary for evaluating chronic myelogenous leukemia. For example, based on the results of testing, a physician may assess and/or diagnose whether or not a patient is affected by chronic myelogenous leukemia, whether or not TKI treatment of chronic myelogenous leukemia should be continued, or whether or not chronic myelogenous leukemia has relapsed after discontinuation of TKI treatment, and may determine the appropriate course of treatment. In other words, “testing” is clearly distinguished from the act carried out by the physician. Also, the term “testing” as used herein includes not only application for aiding in diagnosis, but also application for in vitro study and research.

The phrase “method for testing chronic myelogenous leukemia”, as used herein, includes methods of aiding in diagnosis of whether or not chronic myelogenous leukemia is suffered, methods of aiding in monitoring the therapeutic effect on chronic myelogenous leukemia, methods of aiding in classifying a patient in need of treatment for chronic myelogenous leukemia, and methods of aiding in predicting relapse of chronic myelogenous leukemia. Preferably, the testing method of the invention is a method of aiding in classifying a patient in need of treatment for chronic myelogenous leukemia or a method of aiding in predicting relapse of chronic myelogenous leukemia.

When the testing method of the invention is a method of aiding in classifying a patient in need of treatment for chronic myelogenous leukemia, the classification of the patient may be carried out, for example, by a method comprising steps of: comparing the number of ADAM8-expressing cells or the expression level of the ADAM8 gene, measured by the testing method of the invention, with a reference value; and if the number of ADAM8-expressing cells or the expression level of the ADAM8 gene is higher than the reference value (for example, statistically significantly higher), classifying the patient as being in need of treatment for chronic myelogenous leukemia.

When the testing method of the invention is a method of aiding in predicting relapse of chronic myelogenous leukemia, the prediction of relapse may be carried out, for example, by a method comprising steps of: comparing the number of ADAM8-expressing cells or the expression level of the ADAM8 gene, measured by the testing method of the invention, with a reference value; and if the number of ADAM8-expressing cells or the expression level of the ADAM8 gene is higher than the reference value (for example, statistically significantly higher), predicting that the chronic myelogenous leukemia will relapse.

The “reference value” for the patient classifying method or relapse predicting method may be determined based on the number of ADAM8-expressing cells in a sample taken from a healthy person or the expression level of the ADAM8 gene in blood cells in a sample taken from a healthy person, or based on the number of ADAM8-expressing cells in a sample taken from a subject without relapse after discontinuation of TKI treatment (for example, a subject without relapse for at least 2 years after discontinuation of TKI treatment) or based on the expression level of the ADAM8 gene in blood cells in a sample taken from a subject without relapse after discontinuation of TKI treatment. The reference value may also be the mean value or median value of the number of ADAM8-expressing cells in a sample obtained from multiple subjects, or the mean value or median value of the expression level of the ADAM8 gene in blood cells in a sample obtained from multiple subjects.

The term “subject”, as used herein, is not particularly restricted so long as it is a mammal (for example, a human, monkey, mouse, rat, dog, cat, rabbit, cow, horse, sheep, goat, pig, deer or the like), but it is preferably a human.

The “sample obtained from a subject” for the testing method of the invention includes tissues and body fluids derived from a subject that contain blood cells. The sample may include culture solution containing blood cells obtained by hematopoietic differentiation of induced pluripotent stem cells (iPSCs) obtained by reprogramming cells obtained from a subject. The sample used for the testing method of the invention is preferably peripheral blood or bone marrow fluid obtained from a subject.

As used herein, the term “blood cells” includes all forms of blood cells that exist throughout the entire process of differentiation, from hematopoietic stem cell progenitor cells and hematopoietic stem cells to final differentiation in the peripheral blood. While not particularly restrictive, this includes leukocytes (neutrophils, eosinophils, basophils, lymphocytes (T cells and B cells), monocytes and dendritic cells), hematopoietic cells, hematopoietic stem cells, hematopoietic progenitor cells and peripheral blood mononuclear cells (PBMCs).

The blood cells for the testing method of the invention are preferably CD34-positive cells. The term “CD34-positive”, as used herein, means expressing CD (cluster of differentiation) 34 antigen on the cell surface. This antigen is a marker of hematopoietic stem cells and hematopoietic progenitor cells, and is lost upon differentiation. CD34-positive cells (clusters) are cell populations containing greater amounts of hematopoietic stem cells and hematopoietic progenitor cells. The term “positive” may be indicated as “+”, and “negative” may be indicated as “−”.

According to one embodiment, the blood cells for the testing method of the invention are blood cells with a ratio of no greater than 0.1% for the expression level of the BCR-ABL gene with respect to the expression level of the ABL gene, and preferably they are CD34-positive cells with a ratio of no greater than 0.1% for the expression level of the BCR-ABL gene with respect to the expression level of the ABL gene.

According to one embodiment, the subject for the testing method of the invention is a subject undergoing treatment with a tyrosine kinase inhibitor.

The term “tyrosine kinase inhibitor”, as used herein, includes all therapeutic agents that act as selective or non-selective inhibitors of receptor-type and/or non-receptor-type tyrosine kinases. While not particularly restrictive, examples include Imatinib, Gefitinib, Erlotinib, Osimertinib, Afatinib, Dasatinib, Bosutinib, Vandetanib, Sunitinib, Axitinib, Pazopanib, Lenvatinib, Lapatinib, Nintedanib, Nilotinib, Ibrutinib and Ponatinib, among which Imatinib is preferred.

MMR (major molecular response) is known as an assessment standard for therapeutic effect on chronic myelogenous leukemia. In BCR-ABL evaluation, for example, a subject is assessed as MMR when the ratio is no greater than 0.1% for the expression level of the BCR-ABL gene with respect to the expression level of the ABL gene. When BCR-ABL has not been detected at least two consecutive times in BCR-ABL evaluation, the subject is assessed as MUL (molecular undetectable leukemia) (or CMR (complete molecular response)). BCR-ABL evaluation may be carried out by a method known to those skilled in the art (for example, the method described in NPL 2).

According to one embodiment, the subject for the testing method of the invention is a subject that has reached MMR or MUL by treatment with a tyrosine kinase inhibitor.

As used herein, “detection” is qualitative or quantitative determination of the presence of cells, mRNA and protein. Also as used herein, “measurement” means quantitatively determining a cell count, mRNA expression level and/or protein expression level.

According to one embodiment, “detecting expression of the ADAM8 gene” in the testing method of the invention includes detecting cells that express the ADAM8 gene and/or measuring the number of those cells. Such detection and/or measurement is not particularly restricted, and may be carried out by flow cytometry, for example.

According to another embodiment, “detecting expression of the ADAM8 gene” in the testing method of the invention includes detecting mRNA transcribed from the ADAM8 gene and/or measuring the amount of that mRNA. An mRNA sequence from human ADAM8 is listed as SEQ ID NO: 1. Such detection and/or measurement is not particularly restricted, and for example, it may be carried out by a method using Northern blotting, reverse transcriptase PCR (RT-PCR; e.g. quantitative RT-PCR), real-time PCR, in situ hybridization (for example, quantitative in situ hybridization), or a microarray (for example, an oligonucleotide array or gene chip).

Measurement of the amount of mRNA may be measurement using a nucleotide probe or primer that hybridizes to mRNA (or cDNA or cRNA for the mRNA). The base length of the probe or primer is 10 to 50 nucleotides and preferably 15 to 25 nucleotides. When an amount of mRNA is to be measured using RT-PCR for the testing method of the invention, the cDNA obtained by reverse transcription of the RNA extracted from the subject sample may be amplified using primers (for example, the primers represented by SEQ ID NO: 3 and 4) to measure the amount of mRNA.

According to another embodiment, “detecting expression of the ADAM8 gene” in the testing method of the invention includes detecting a protein encoded by the ADAM8 gene and/or measuring the amount of that protein. The amino acid sequence of a human ADAM8 protein is listed as SEQ ID NO: 2. Such detection and/or measurement is not particularly restricted, and for example, it may be carried out by an immunochemical method using highly specific polyclonal antibodies or monoclonal antibodies for the protein. Immunochemical methods are not particularly restricted and include Western blotting, EIA, RIA and chemiluminescent immunoassay (CLIA). The antibody used for detection and/or measurement may be commercially available antibody that is already confirmed to have specificity, or it may be newly constructed by a standard antibody construction method based on sequence data for ADAM8.

### <Isolation Method of the Invention>

According to a second aspect, the invention relates to a method for isolating chronic myelogenous leukemia cells having resistance to a tyrosine kinase inhibitor from blood cells in a sample obtained from a subject, the method comprising steps of:

(1) detecting expression of the ADAM8 gene in blood cells in a sample; and

(2) isolating chronic myelogenous leukemia cells having resistance to the tyrosine kinase inhibitor, based on the detection results of step (1). This will hereunder also be referred to as “isolation method of the invention”.

The terms “subject”, “sample”, “blood cell” and “detecting expression of the ADAM8 gene” for the isolation method of the invention are the same as explained for the first aspect.

As used herein, the phrase “chronic myelogenous leukemia cells having resistance to a tyrosine kinase inhibitor” (hereunder also referred to as “TKI-resistant CML cells”) are chronic myelogenous leukemia cells having resistance to a tyrosine kinase inhibitor, and whose viability cannot be significantly reduced by administration of the tyrosine kinase inhibitor. While not particularly restrictive, these may be TKI-resistant CD34-positive cells from a CML patient, for example. They may also be TKI-resistant CD34-positive CD38-positive cells (progenitor cell population) and/or TKI-resistant CD34-positive CD38-negative cells (stem cell population) from a CML patient. The TKI-resistant CML cells may include chronic myelogenous leukemia stem cells (hereunder also referred to as “CML stem cells”). CML stem cells are cells that have developed into cancer stem cells while retaining the stemness of hematopoietic stem cells. As used herein, “TKI-resistant CML cells” include the TKI-resistant CML cell model prepared in Example 1 described below.

According to one embodiment, the isolation method of the invention comprises measuring the number of ADAM8 gene-expressing cells among blood cells in a sample, and isolating the cells if the cell count is higher (for example, statistically significantly higher) than a reference value. According to another embodiment, the isolation method of the invention comprises measuring the expression level of the ADAM8 gene in blood cells in a sample, and isolating the cells if the gene expression level is higher (for example, statistically significantly higher) than a reference value. The reference value is the same as explained for the first aspect.

According to the method of the invention, the cell isolating step may employ a cell isolating method known to those skilled in the art. While not particularly restrictive, it may be a method for isolating cells by flow cytometry using an antibody for ADAM8 protein (FACS), or a method of using magnetic beads supporting the antibody to collect and isolate the cells by magnetism (MACS).

### <Kit>

According to a third aspect, the invention relates to a kit for testing chronic myelogenous leukemia, comprising a detection reagent for mRNA transcribed from the ADAM8 gene or a detection reagent for a protein encoded by the ADAM8 gene. The kit is used in the testing method of the invention and the isolation method of the invention, for example.

When the object of detection is mRNA transcribed from the ADAM8 gene, the kit of the invention may include as detection reagents the primers to be used in PCR and the necessary reagents for the PCR reaction. Alternatively, it may also include cDNA and cRNA for mRNA, or portions thereof, as probes to be used for Northern blotting. The detection reagents may be the primers listed as SEQ ID NO: 3 and 4, for example.

When the object of detection is a protein encoded by the ADAM8 gene, the kit of the invention may include, as “detection reagents” to be included in the testing kit of the invention, the specific primary antibody and secondary antibody to be used in the immunochemical method and the detection reagent for the enzyme bound to the secondary antibody. For example, the detection reagent may be an antibody for human ADAM8 by Miltenyi Biotec, which is used in the Examples.

### <Composition for Lowering TKI Resistance>

According to a fourth aspect, the invention relates to a composition for lowering tyrosine kinase inhibitor resistance in chronic myelogenous leukemia, comprising an ADAM8 gene expression inhibitor or an ADAM8 activity inhibitor. This will hereunder also be referred to as “composition for lowering TKI resistance of the invention”. The phrase “lowering tyrosine kinase inhibitor (TKI) resistance in chronic myelogenous leukemia (CML)” means partially lowering TKI resistance or completely eliminating said resistance, in a subject with TKI-resistant CML cells.

The effect of the composition for lowering TKI resistance of the invention can be confirmed based on the cell viability of TKI-resistant CML cells (for example, cells isolated by the method of the second aspect or the TKI-resistant CML cell model prepared in Example 1) when treated with TKI after treatment with the composition for lowering TKI resistance of the invention, or based on a lower (for example, statistically significantly lower) cell viability compared to a control, when comparison is with the cell viability of the control. The cell viability of the control is the cell viability when TKI-resistant CML cells are treated with a tyrosine kinase inhibitor, without treatment with the composition for lowering TKI resistance of the invention.

### (ADAM8 Activity Inhibitor)

As used herein, “ADAM8 activity inhibitor” means a natural or synthesized compound that exhibits biological effectiveness for directly and/or indirectly inhibiting or significantly suppressing activity (for example, metalloprotease activity or disintegrin activity) of the ADAM8 protein. The ADAM8 activity inhibitor may be a low molecular weight organic substance, aptamer, antibody or antibody fragment, or a combination thereof. It is preferably an antibody or antibody fragment.

As used herein, the term “low molecular weight organic substance” refers to a molecule of approximately the same size as organic molecules commonly used in pharmaceuticals. The size of a low molecular weight organic substance as an ADAM8 activity inhibitor that may be used for the invention is in the range of preferably no greater than about 5,000 Da, more preferably no greater than about 2,000 Da and most preferably no greater than about 1,000 Da. An example of a low molecular weight organic substance that may be used as an ADAM8 activity inhibitor is GM6001 ((2R)—N⁴-Hydroxy-N¹-[(1S)-2-(1H-indol-3-ylmethyl)-2-(methylamino)-2-oxoethyl]-2-(2-methylpropyl)butanediamide; CAS No. 142880-36-2; molecular weight: 388.47).

As used herein, “aptamer” refers to a synthetic DNA or RNA molecule or peptide molecule that has the ability to specifically bind to a target substance, and it can be chemically synthesized rapidly in vitro. The aptamer to be used for the invention can bind to ADAM8 protein and inhibit activity of the ADAM8 protein. The aptamer to be used for the invention can be obtained, for example, by selection by repetitive in vitro binding to various molecular targets such as small molecules, proteins and nucleic acids, using the SELEX method (see Tuerk C., Gold L., Science, 1990, 249(4968), 505-510; Ellington A D, Szostak J W., Nature, 1990, 346(6287):818-822; U.S. Pat. No. 6,867,289; U.S. Pat. No. 5,567,588; and U.S. Pat. No. 6,699,843).

As used herein, an “antibody or antibody fragment” may be any antibody such as a human-derived antibody, mouse-derived antibody, rat-derived antibody, rabbit-derived antibody or goat-derived antibody, so long as it binds to ADAM8 protein and inhibits ADAM8 activity, and polyclonal or monoclonal antibodies thereof may also be complete or shortened (for example, F(ab′)2, Fab′, Fab or Fv fragment) antibodies, chimeric antibodies, humanized antibodies or fully human antibodies. As used herein, “antibody fragment” is an F(ab′)2, Fab′, Fab or scFv antibody fragment, which can be obtained by treatment of an antibody with a protease enzyme, or in some cases reduction.

The antibody or antibody fragment to be used for the invention may be produced by a known method for producing antibodies or antiserum, using ADAM8 protein or a portion thereof as the antigen. The ADAM8 protein or portion thereof can be prepared by a known protein expression method and purification method. An example of an ADAM8 protein is human ADAM8 consisting of the amino acid sequence listed as SEQ ID NO: 2, but this is not limitative. Various biologically-derived ADAM8 proteins can be suitably used as immunogens. Their amino acid sequences are readily available from public databases (such as Protein Data Bank). The antibody or antibody fragment to be used for the invention can also be constructed by the phage display method (see FEBS Letter, 1998, Vol. 441, p. 20-24, for example).

### (ADAM8 Gene Expression Inhibitor)

As used herein, “ADAM8 gene expression inhibitor” means a natural or synthesized compound that has biological effectiveness for directly and/or indirectly inhibiting or significantly suppressing expression of an ADAM8 gene. Examples include antisense oligonucleotides, RNAi-inducible nucleic acids, microRNA (miRNA), ribozymes, genome editing nucleic acids and their expression vectors, low molecular weight organic substances, aptamers, antibodies, antibody fragments, and combinations thereof. Preferred are siRNA, shRNA or antisense oligonucleotides.

As used herein, “antisense oligonucleotide” is DNA or RNA having a nucleotide sequence (a sequence of 5 to 100 nucleotides, for example) that is complementary to functional RNA (sense RNA) such as messenger RNA (mRNA), and that forms a double strand with the sense RNA, having the function of inhibiting synthesis of the protein that is normally carried out by the sense RNA. According to the invention, an antisense oligonucleotide containing an antisense RNA or DNA molecule binds with ADAM8 mRNA, thereby inhibiting its translation to protein. This allows expression level of ADAM8 protein to be lowered. The antisense oligonucleotide may also be modified, so long as there is not obstruction to its function. Methods of synthesizing antisense oligonucleotides are well known in the technical field, and for example, synthesis may be easily carried out using a commercially available DNA synthesizer. The sequence of the target mRNA may be the nucleotide sequence of mRNA from human ADAM8, listed as SEQ ID NO: 1, but this is not limitative. The nucleotide sequences of ADAM8-derived mRNA of various organisms are readily available from public databases (such as GenBank).

As used herein, “RNAi-inducible nucleic acid” refers to a polynucleotide that is capable of inducing RNA interference (RNAi) by being introduced into cells, and it may usually be RNA or DNA, or a chimeric molecule of RNA and DNA, comprising 19 to 30 nucleotides, preferably 19 to 25 nucleotides and more preferably 19 to 23 nucleotides, optionally with desired modification. The RNAi may be produced for the mRNA, or it may be transcribed RNA just before processing, i.e. RNA having a nucleotide sequence including the exon, intron, 3′-untranslated region and 5′-untranslated region. The RNAi method that may be used for the invention may be induction of RNAi by a method such as (1) directly introducing short double-stranded RNA (siRNA) into cells, (2) incorporating short hairpin RNA (shRNA) into different expression vectors and introducing the vectors into cells, or (3) creating a vector that expresses siRNA by inserting short double-stranded DNA corresponding to the siRNA, between promoters in a vector having two promoters running in opposite directions, and introducing the vector into cells. The RNAi-inducible nucleic acid may include siRNA, shRNA or miRNA capable of cleaving ADAM8 mRNA or suppressing its function, and such RNAi-inducible nucleic acid may be directly introduced using liposomes or the like, or it may be introduced using an expression vector that induces the RNAi nucleic acid. The sequence of the target RNA may be the nucleotide sequence of mRNA from human ADAM8, listed as SEQ ID NO: 1, but this is not limitative. The nucleotide sequences of ADAM8-derived mRNA of various organisms are readily available from public databases (such as GenBank).

The RNAi-inducible nucleic acid for ADAM8 to be used for the invention can be synthesized using known chemical synthesis techniques, based on the RNA sequence for ADAM8 which is the target of the RNAi-inducible nucleic acid. For example, it may be chemically synthesized using a DNA (/RNA) automatic synthesizer utilizing DNA synthesis technology such as the solid phase phosphoramidite method, or it may be synthesized by consignment to an siRNA-related contracted synthesis company (such as Life Technologies). According to an embodiment of the invention, the siRNA to be used for the invention can be derived from short-hairpin-type double stranded RNA (shRNA) as the precursor, via processing with a dicer, which is an intracellular RNase. The RNAi-inducible nucleic acid to be used for the invention may be the shRNA of SEQ ID NO: 45 to 47, for example.

As used herein, “microRNA (miRNA)” is a single-stranded RNA molecule with a length of 21 to 25 bases, and it contributes to regulation of post-transcriptional expression of genes in eukaryotes. The miRNA generally recognizes 3′UTR in mRNA, inhibiting translation of target mRNA and inhibiting protein production. Thus, miRNA that can directly and/or indirectly lower expression levels of the ADAM8 gene is also within the scope of the present invention.

As used herein, “ribozyme” is a general term for enzymatic RNA molecules that can catalyze specific cleavage of RNA. Ribozymes with different activities exist, and research focusing on ribozymes as RNA-cleaving enzymes has made it possible to design ribozymes that cleave RNA in a site-specific manner. Ribozymes include large ones of 400 or more nucleotides such as M1 RNA, which are included in group I introns or RNase P, but some have active domains of about 40 nucleotides, known as hammerhead types or hairpin types. By using a ribozyme to specifically cleave an ADAM8 gene transcription product it is possible to inhibit expression of the ADAM8 gene.

As used herein, “genome editing nucleic acid” refers to a nucleic acid used for editing of a desired gene in a system utilizing a nuclease that is used for gene targeting. Nucleases used for gene targeting include known nucleases, and also novel nucleases to be used for future gene targeting. For example, known nucleases include CRISPR/Cas9 (Ran, F. A., et al., Cell, 2013, 154, 1380-1389), TALEN (Mahfouz, M., et al., PNAS, 2011, 108, 2623-2628) and ZFN (Urnov, F., et al., Nature, 2005, 435, 646-651).

The CRISPR/Cas9 system can introduce double-stranded cuts into desired sites of DNA. Using a CRISPR/Cas9 system requires at least 3 elements: a protospacer adjacent motif (PAM sequence), guide RNA (gRNA) and a Cas protein (Cas, Cas9).

A gRNA molecule is designed so as to have the sequence complementary to a target site adjacent to the PAM sequence (5′-NGG), and it is introduced into the desired cells together with the Cas protein. The transferred gRNA and the Cas protein form a complex. The gRNA binds to the target sequence in the genome, and the Cas protein cleaves the double strand of the target genomic DNA by its nuclease activity.

This is followed by homology directed repair (HDR) or non-homologous end joining (NHEJ) in the cells that have undergone double-stranded cleavage by the nuclease. When suitable DNA fragments (for example, HDR repair templates) are present in the cells, homologous recombination takes place and it is possible to accomplish modification such as deletion, insertion or destruction in the target genome. When an HDR repair template is not present, several nucleotide deletions or additions may take place during the course of NHEJ. This produces a frameshift in the region coding for the protein, destroying the reading frame of the protein or introducing an immature stop codon, making it possible to knock-out the protein as a result.

The genome editing nucleic acid to be used for the invention may be gRNA targeting the ADAM8 gene, or it may be a vector expressing the gRNA. According to another embodiment, the genome editing nucleic acid may also include a nucleic acid expressing the nuclease to be used for the gene targeting. The gRNA and the nuclease to be used for gene targeting (preferably Cas protein) may be encoded in the same vector, or separately coding vectors may be used. According to another embodiment, the genome editing nucleic acid may further include HDR repair template nucleic acid. The genome editing nucleic acid may be a plasmid vector or a viral vector. The genome editing nucleic acid can be introduced into arbitrary cells by a publicly known method, with no particular restrictions.

The ADAM8 gene expression inhibitor may be provided as an expression vector having the antisense oligonucleotide, RNAi-inducible nucleic acid, microRNA (miRNA) and ribozyme or genome editing nucleic acid encoded in a desired vector. According to the invention, the vector to be used for expression of the ADAM8 gene expression inhibitor is not particularly restricted, and a publicly known one may be selected as appropriate. For example, it may be a plasmid vector, cosmid vector, phasmid vector, viral vector, or artificial chromosomal vector. The ADAM8 expression inhibitor can be introduced into the vector by using publicly known gene recombinant technology, with no particular restrictions.

### (Formulation)

The composition for lowering TKI resistance of the invention can be formulated by appropriately combining the ADAM8 gene expression inhibitor or ADAM8 activity inhibitor as active ingredients, with a pharmaceutically acceptable carrier or additives. Specifically, it may be prepared as an oral drug such as tablets, coated tablets, pills, powder, granules, capsules, a liquid drug, suspending agent or emulsion; or as a parenteral agent such as an injection, infusion, suppository, ointment or patch. The mixing proportion of the carrier or additives may be appropriately set based on ranges commonly employed in the field of pharmaceuticals. The carrier or additives that may be combined are not particularly restricted, and examples include various carriers such as water, physiological saline, other aqueous solvents and aqueous or oil bases; and various additives such as excipients, binders, pH regulators, disintegrators, absorption accelerators, lubricants, coloring agents, taste correctives and aromatics.

### (Subject of Administration)

The subject to be administered the composition for lowering TKI resistance of the invention is a human or a non-human mammal. A non-human mammal may be, specifically, a monkey, mouse, rat, dog, cat, rabbit, cow, horse, sheep, goat, pig or deer. The subject of administration is preferably a human, more preferably a human patient suffering from CML, and even more preferably a human patient with TKI-resistant CML cells. The composition for lowering TKI resistance of the invention may be used in vitro for cells of a human or non-human mammal.

### (Route of Administration and Dose)

The route of administration of the composition for lowering TKI resistance of the invention may be oral or parenteral. Specifically, parenteral administration includes injection, nasal administration, transpulmonary administration and transdermal administration. Examples of injection include intravenous injection, intramuscular injection, intraperitoneal injection and hypodermic injection. The composition can be systemically or locally administered by injection. The method of administration may also be appropriately selected depending on the age and symptoms of the patient. The dose of the ADAM8 gene expression inhibitor or ADAM8 activity inhibitor, as the active ingredient, may be a dose in a range of 0.0001 mg to 1,000 mg per 1 kg of body weight for each administration. Alternatively, the dose of the active ingredient may be selected in the range of 0.001 mg/body to 100,000 mg/body per patient. However, the dose of the composition for lowering TKI resistance of the invention is not limited to these ranges.

### <Composition for Treatment of CML>

According to a fifth aspect, the invention relates to a composition for treatment of chronic myelogenous leukemia comprising a tyrosine kinase inhibitor, wherein the composition is administered to a subject in combination with an ADAM8 gene expression inhibitor or ADAM8 activity inhibitor. This will hereunder also be referred to as “composition for treatment of CML of the invention”.

Administration of an ADAM8 gene expression inhibitor or ADAM8 activity inhibitor to a subject suffering from chronic myelogenous leukemia lowers resistance against tyrosine kinase inhibitors. As a result, the tyrosine kinase inhibitor can more effectively act in the subject. The effect of the tyrosine kinase inhibitor can also be augmented.

In the composition for treatment of CML of the invention, “administered to a subject in combination” means that the composition for treatment of CML of the invention is administered before the ADAM8 gene expression inhibitor or ADAM8 activity inhibitor is administered to the subject, or after administration of the inhibitor, or simultaneously with administration of the inhibitor. Preferably, the composition for treatment of CML of the invention is administered after administering the ADAM8 gene expression inhibitor or ADAM8 activity inhibitor to the subject.

The “ADAM8 gene expression inhibitor” and “ADAM8 activity inhibitor” to be administered in combination with the composition for treatment of CML of the invention, and the “formulation” and “route of administration” of the composition for treatment of CML of the invention, are the same as explained for the fourth aspect.

The subject to which the composition for treatment of CML of the invention is administered is a human or a non-human mammal. A non-human mammal may be, specifically, a monkey, mouse, rat, dog, cat, rabbit, cow, horse, sheep, goat, pig or deer. The subject of administration is preferably a human, more preferably a human patient suffering from CML, and even more preferably a human patient with TKI-resistant CML cells.

The dosage for the composition for treatment of CML of the invention may be a dose such that the tyrosine kinase inhibitor, which is the active ingredient, is in the range of 0.0001 mg to 1,000 mg per 1 kg of body weight per administration. Alternatively, the dosage of the tyrosine kinase inhibitor as the active ingredient may be in the range of 0.001 mg/body to 100,000 mg/body per patient. However, the dosage of the composition for treatment of CML of the invention is not limited to these ranges.

### <Screening Method>

According to a sixth aspect, the invention relates to a method for screening an agent for lowering tyrosine kinase inhibitor resistance in chronic myelogenous leukemia, wherein ADAM8 gene expression inhibition or ADAM8 activity inhibition is used as the marker. This will hereunder also be referred to as “screening method of the invention”.

### (Method Using ADAM8 Gene-Expressing Cells)

The screening method of the invention may be carried out by a procedure comprising steps of:

contacting ADAM8 gene-expressing cells with a test substance;

measuring the expression level of the ADAM8 gene or ADAM8 activity in the cells;

comparing the expression level of the ADAM8 gene or ADAM8 activity measured in the previous step with a control expression level or a control activity, the control expression level being the expression level of the ADAM8 gene in ADAM8 gene-expressing cells that have not been contacted with the test substance and the control activity being ADAM8 activity in ADAM8 gene-expressing cells that have not been contacted with the test substance; and

selecting a test substance that lowers the expression level of the ADAM8 gene or ADAM8 activity in comparison with the control expression level or control activity.

The ADAM8 gene-expressing cells to be used for this method are not particularly restricted so long as they can be used for screening according to the invention, and they may be TKI-resistant CML cells isolated by the second aspect, for example. They may also be the TKI-resistant CML cell model prepared in Example 1.

The contact time and contact temperature between the ADAM8 gene-expressing cells and the test substance in the method of the invention are not particularly restricted and may be selected as appropriate.

The expression level of the ADAM8 gene for this method may be measured by the method described for the first aspect.

Evaluation of the ADAM8 activity is not particularly restricted, and it may be carried out, for example, by evaluating the metalloprotease activity of the ADAM8 protein. ADAM8 protein is known to degrade CD23 protein by metalloprotease activity. ADAM8 protein activity can be measured by simultaneously expressing CD23 protein in ADAM8 gene-expressing cells and measuring the amount of CD23 protein degraded. (See U. Schlomann et al, “ADAM8 as a drug target in pancreatic cancer”, Nature communications, 6, 6175(2015), for example).

When the expression level of the ADAM8 gene in ADAM8 gene-expressing cells contacted with a test substance is compared with a control expression level and the expression level is lower (for example, statistically significantly lower) than the control, the test substance may be assessed and selected as being an agent for lowering tyrosine kinase inhibitor resistance in chronic myelogenous leukemia. Preferably, the test substance is assessed and selected as being an agent for lowering tyrosine kinase inhibitor resistance in chronic myelogenous leukemia when the expression level of the ADAM8 gene in the ADAM8 gene-expressing cells that have been contacted with the test substance is no greater than 90%, no greater than 80%, no greater than 70%, no greater than 60% or no greater than 50% of the control expression level.

Alternatively, the test substance may be assessed and selected as being an agent for lowering tyrosine kinase inhibitor resistance in chronic myelogenous leukemia when the ADAM8 activity in ADAM8 gene-expressing cells that have been contacted with a test substance is lower (for example, statistically significantly lower) than the control activity, when compared with the control. Preferably, the test substance is assessed and selected as being an agent for lowering tyrosine kinase inhibitor resistance in chronic myelogenous leukemia when the ADAM8 activity in the ADAM8 gene-expressing cells that have been contacted with the test substance is no greater than 90%, no greater than 80%, no greater than 70%, no greater than 60% or no greater than 50% of the control activity.

### (Method Using ADAM8 Protein)

The screening method of the invention may be carried out by a procedure comprising steps of:

contacting ADAM8 with a test substance;

measuring the ADAM8 activity;

comparing the ADAM8 activity measured in the previous step with a control activity, the control activity being the ADAM8 activity in the absence of contact with the test substance; and

selecting a test substance that lowers the ADAM8 activity in comparison with the control activity.

The contact time and contact temperature between the ADAM8 protein and the test substance in the method using ADAM8 protein are not particularly restricted, and may be selected as appropriate.

Evaluation of the ADAM8 protein for the method using ADAM8 protein is not particularly restricted, and it may be carried out, for example, by evaluating the metalloprotease activity of the ADAM8 protein. For example, after setting the optimal conditions for combining the ADAM8 protein and CD23 protein, the amount of degraded CD23 protein may be measured to determine the ADAM8 protein activity. Alternatively, the ADAM8 protein activity can be evaluated based on the disintegrin activity of the ADAM8 protein (see Fourie, A. M. et al., “Catalytic activity of ADAM8, ADAM15, and MDC-L (ADAM28) on synthetic peptide substrates and in ectodomain cleavage of CD23”, J. Biol. Chem. 278, 30469-30477 (2003), for example).

When the activity of ADAM8 contacted with a test substance is compared with a control activity and the expression level is lower (for example, statistically significantly lower) than the control, the test substance may be assessed and selected as being an agent for lowering tyrosine kinase inhibitor resistance in chronic myelogenous leukemia. Preferably, the test substance is assessed and selected as being an agent for lowering tyrosine kinase inhibitor resistance in chronic myelogenous leukemia when the activity of ADAM8 that has been contacted with the test substance is no greater than 90%, no greater than 80%, no greater than 70%, no greater than 60% or no greater than 50% of the control activity.

The screening method of the invention may further include a step of treating TKI-resistant CML cells with a selected test substance and then with a tyrosine kinase inhibitor, and confirming whether or not resistance has been lowered. The TKI-resistant CML cells to be used in this step may be cells isolated by the method of the second aspect of the invention, or they may be the TKI-resistant CML cell model prepared in Example 1. The method of confirming lowered resistance is not particularly restricted, and for example, the cell viability of TKI-resistant CML cells when treated with a selected test substance and the treated with tyrosine kinase inhibitor may be compared with a control cell viability, and when the cell viability is lower (for example, statistically significantly lower) than the control, and preferably when the cell viability is no greater than 90%, no greater than 80%, no greater than 70%, no greater than 60% or no greater than 50% than the control, the selected test substance may be confirmed to be an agent for lowering tyrosine kinase inhibitor resistance in chronic myelogenous leukemia. The control cell viability is the cell viability when TKI-resistant CML cells have been treated with a tyrosine kinase inhibitor, without treatment with the selected test substance.

As explained above, according to one or more embodiments of the invention it is possible to detect and isolate TKI-resistant CML cells in a more high sensitive manner than by the prior art methods. Moreover, according to one or more embodiments of the invention, it is possible to efficiently carry out diagnosis of CML, monitoring of CML, classification of patients in need of TKI treatment for CML, and prediction of CML relapse, using ADAM8 expression in blood cells as the marker. Moreover, according to one or more embodiments of the invention, it is possible to lower TKI resistance in CML.

All of the publications mentioned throughout the present specification are incorporated herein in their entirety by reference. The technical scope of the invention is limited solely by the description in the Claims. Modifications of the invention, such as additions, deletions or substitutions to the constituent features of the invention, are possible so long as the gist of the invention is maintained.

### EXAMPLES

The present invention will now be explained in further detail with reference to examples, with the understanding that the scope of the invention is naturally not limited to the examples or their descriptions.

### Patient Samples

Primary samples from bone marrow of all patients used in the Examples were obtained after informed consent. All of the Examples using human cells were reviewed and approved by the Institutional Review Board (IRB) of the University of Tokyo. The CML-CP patient samples were chosen which determined no more than t(9;22) (q34;q11) chromosomal translocation on G-banded chromosome analysis. Mononuclear cells (MNCs) were isolated by centrifugation through a Ficoll gradient. The details for the patient samples used in the Examples were as follows.

TABLE 1
Samples used for establishing CML-iPSCs

| | | | | | | Molecular response |
|---|---|---|---|---|---|---|
| | | | | Ph1 | BCR-ABL | after continuing TKI |
| Established iPSCs | Case No. | Age | Gender | (G-band) | type | treatment for 2 years |
| CML-iPSC Pt. 1 | CML-CP26 | 44 | Male | 20/20 (100%) | P210(b3a2) | MMR |
| CML-iPSC Pt. 2 | CML-CP4 | 22 | Male | 20/20 (100%) | P210(b3a2) | MMR |

TABLE 2
Samples used in flow cytometry assay and cell viability assay

| | | | Ph1 | | |
|---|---|---|---|---|---|
| Case No. | Age | Gender | (G-band) | FISH BCR-ABL | BCR-ABL type |
| CML-CP 1 | 66 | Male | 20/20 (100%) | N/A | P210 |
| CML-CP 2 | 59 | Female | 20/20 (100%) | 180/200 (90%) | P210(b3a2) |
| CML-CP 3 | 66 | Male | 20/20 (100%) | 193/200 (96.5%) | P210(b3a2) |
| CML-CP 4 | 22 | Male | 20/20 (100%) | 171/200 (85.5%) | P210(b3a2) |
| CML-CP 5 | 54 | Female | 20/20 (100%) | 194/200 (94%) | P210(b3a2) |
| CML-CP 6 | 40 | Male | 20/20 (100%) | N/A | P210(b3a2) |
| CML-CP 7 | 66 | Male | 20/20 (100%) | 188/200 (94%) | P210(b2a2) |
| CML-CP 8 | 62 | Female | 19/20 (95%) | N/A | P210(b3a2) |
| CML-CP 9 | 56 | Male | 20/20 (100%) | N/A | P210(b2a2) |
| CML-CP 10 | 65 | Male | 20/20 (100%) | 192/200 (96%) | P210(b2a2) |
| CML-CP 11 | 61 | Female | 20/20 (100%) | 196/200 (98%) | P210(b3a2) |
| CML-CP 12 | 58 | Male | 19/20 (95%) | N/A | P210(b3a2) |
| CML-CP 13 | 33 | Male | 16/20 (80%) | N/A | P210(b3a2) |
| CML-CP 14 | 51 | Female | 17/20 (80%) | N/A | P210(b2a2) |
| CML-CP 15 | 65 | Male | 20/20 (100%) | N/A | P210(b3a2) |
| CML-CP 16 | 42 | Male | 20/20 (100%) | N/A | P210(b2a2) |
| CML-CP 17 | 75 | Female | 20/20 (100%) | 192/200 (96%) | P210(b2a2) |
| CML-CP 18 | 54 | Female | 20/20 (100%) | N/A | P210(b3a2) |
| CML-CP 19 | 63 | Female | 20/20 (100%) | 186/200 (93%) | P210(b3a2) |
| CML-CP 20 | 51 | Female | 19/20 (95%) | 192/200 (96%) | P210(b3a2) |
| CML-CP 21 | 35 | Male | 20/20 (100%) | N/A | P210 |
| CML-CP 22 | 86 | Male | 20/20 (100%) | 193/200 (96.5%) | P210(b2a2) |
| CML-CP 23 | 23 | Female | 20/20 (100%) | N/A | P210(b3a2) |

TABLE 3
Samples used in limiting dilution assay

| | | | | TKI treatment | Molecular |
|---|---|---|---|---|---|
| Case No. | Age | Gender | TKI | period | response |
| CML-CP 16 | 42 | Male | Dasatinib | 30 months | MMR |
| CML-CP 24 | 67 | Female | Dasatinib | 6 months | MMR |
| CML-CP 25 | 49 | Female | Imatinib | 24 months | MUL |

### RNA Extraction, RT-PCR, Real-Time Quantitative PCR and One-Step Quantitative RT-PCR Analysis

After extraction of total RNA with RNAeasy reagent (QIAGEN), reverse transcription was performed with ReverTra Ace qPCR RT Master Mix (TOYOBO). The primer sequences used for PCR are shown below. Real-time quantitative PCRs were carried out in ABI-7000 Sequencing System with THUNDERBIRD SYBR qPCR Mix, following the manufacturer's instructions (TOYOBO). Each assay was performed in triplicate. 18S rRNA and β-actin were used as endogenous controls.

TABLE 4

| Gene | Use | Forward primer | Reverse primer |
|---|---|---|---|
| ADAM8 | Real-time qPCR | CGATGATGCTGCCTGCGATTG (SEQ ID NO: 3) | CGCAGGTGGAGGGTGAAGTT (SEQ ID NO: 4) |
| IL1RL1 | Real-time qPCR | ATGGGGTTTTGGATCTTAGCAAT (SEQ ID NO: 5) | CACGGTGTAACTAGGTTTTCCTT (SEQ ID NO: 6) |
| MEIS1 | Real-time qPCR | GATATAGCCGTGTTCGCCAAA (SEQ ID NO: 7) | CGGTGGCAGAAATTGTCACAT (SEQ ID NO: 8) |
| BCR-ABL | Real-time qPCR | AACTCCAGACTGTCCACAGCA (SEQ ID NO: 9) | AACGAGCGGCTTCACTCA (SEQ ID NO: 10) |
| 18s RNA | Real-time qPCR | GTAACCCGTTGAACCCCATT (SEQ ID NO: 11) | CCATCCAATCGGTAGTAGCG (SEQ ID NO: 12) |
| OCT3/4 | RT-PCR | CCCCAGGGCCCCATTTTGGTACC | ACCTCAGTTTGAATGCATGGGAGAGC |
| | | (SEQ ID NO: 13) | (SEQ ID NO: 14) |
| OCT3/4 | RT-PCR | CATTCAAACTGAGGTAAGGG (SEQ ID NO: 15) | TAGCGTAAAAGGAGCAACATAG (SEQ ID NO: 16) |
| (transgene) | | | |
| KLF4 | RT-PCR | ACCCATCCTTCCTGCCCGATCAGA | TTGGTAATGGAGCGGCGGGACTTG (SEQ ID NO: 18) |
| | | (SEQ ID NO: 17) | |
| KLF4 | RT-PCR | CCACCTCGCCTTACACATGAAGA | TAGCGTAAAAGGAGCAACATAG (SEQ ID NO: 20) |
| (transgene) | | (SEQ ID NO: 19) | |
| SOX2 | RT-PCR | TTCACATGTCCCAGCACTACCAGA | TCACATGTGTGAGAGGGGCAGTGTGC |
| | | (SEQ ID NO: 21) | (SEQ ID NO: 22) |
| SOX2 | RT-PCR | TTCACATGTCCCAGCACTACCAGA | TTTGTTTGACAGGAGCGACAAT (SEQ ID NO: 24) |
| (transgene) | | (SEQ ID NO: 23) | |
| L-MYC | RT-PCR | GCGAACCCAAGACCCAGGCCTGCTCC | CAGGGGGTCTGCTCGCACCGTGATG |
| | | (SEQ ID NO: 25) | (SEQ ID NO: 26) |
| L-MYC | RT-PCR | GGCTGAGAAGAGGATGGCTAC | TTTGTTTGACAGGAGCGACAAT (SEQ ID NO: 28) |
| (transgene) | | (SEQ ID NO: 27) | |
| LIN28 | RT-PCR | AGCCATATGGTAGCCTCATGTCCGC | TCAATTCTGTGCCTCCGGGAGCAGGGTAGG |
| | | (SEQ ID NO: 29) | (SEQ ID NO: 30) |
| LIN28 | RT-PCR | AGCCATATGGTAGCCTCATGTCCGC | TAGCGTAAAAGGAGCAACATAG (SEQ ID NO: 32) |
| (transgene) | | (SEQ ID NO: 31) | |
| EBNA-1 | RT-PCR | ATCAGGGCCAAGACATAGAGATG | GCCAATGCAACTTGGACGTT (SEQ ID NO: 34) |
| (transgene) | | (SEQ ID NO: 33) | |
| GAPDH | RT-PCR | ACCACAGTCCATGCCATCAC (SEQ ID NO: 35) | TCCACCACCCTGTTGCTGTA (SEQ ID NO: 36) |
| BCR-ABL | Pre- | AGAAGCTTCTCCCTGACATCCG | GGTACCAGGAGTGTTTCTCCAGACTG |
| | amplification | (SEQ ID NO: 37) | (SEQ ID NO: 38) |
| | One-step | TGAAACTCCAGACTGTCC | TCAGACCCTGAGGCTCAAAG (SEQ ID NO: 40) |
| | RT-PCR | (SEQ ID NO: 39) | |
| ACTINB | Pre- | CCAACCGCGAGAAGATGAC | TAGCACAGCCTGGATAGCAA (SEQ ID NO: 42) |
| | amplification | (SEQ ID NO: 41) | |
| | One-step | CGCGAGAAGATGACCCAGAT | CACAGCCTGGATAGCAACGT (SEQ ID NO: 44) |
| | RT-PCR | (SEQ ID NO: 43) | |

### Flow Cytometry (FCM)

Isolation of each cell fraction from hematopoietic cells derived from iPSCs, and primary samples taken from leukemia patients was carried out using FACSAria II and FACSAria III cell sorter (BD Biosciences). Data analysis was performed with a FlowJo (TreeStar, Ashland, Oreg., USA). The following antibodies were used.

TABLE 5
Human

| Epitope | Clone | Fluorophore | Supplier |
|---|---|---|---|
| CD34 | 581 | PE-Cy7 | Biolegend |
| | 4H11 | APC | EBIoscience |
| CD38 | HIT2 | FITC | Biolegend |
| CD43 | DFT1 | PE | BECKMAN |
| | | | COULTER |
| | 4-29-5-10-21 | PerCP-eFluor 710 | EBIoscience |
| CD45 | H130 | APC | Biolegend |
| CD90 | 5.00E+10 | Alexa Fluor 647 | Biolegend |
| ADAM8 | REA331 | PE | Miltenyi Biotec |
| | REA331 | APC | Miltenyi Biotec |
| IgG isotype | MOPC-21 | Alexa Fluor 647 | Biolegend |
| IgG isotype | 679.1 Mc7 | PE | BECKMAN |
| | | | COULTER |

Each of the reagents used in the Examples were commercial products, unless specifically stated otherwise.

### Example 1

### Preparation of TKI-Resistant CML Cell Model

A CML-CP patient primary sample is one of the most effective models for analyzing CML stem cells with TKI resistance. However, since only a small, non-homogeneous amount of CML stem cells can be obtained from a primary sample, it is difficult to efficiently probe novel markers. In order to solve this problem, cells from CML-CP patient bone marrow were reprogrammed to prepare integration-free iPSCs. Hematopoietic differentiation of the iPSCs was then induced to prepare a TKI-resistant CML cell model.

(1) Preparation of iPSCs (Induced Pluripotent Stem Cells) from CML-CP Patient CD34+ Cells.

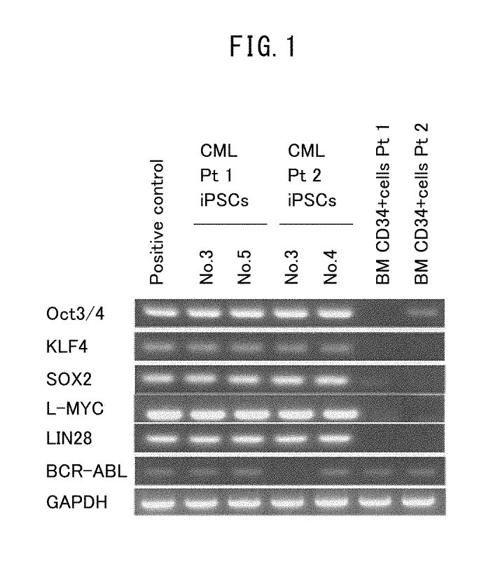

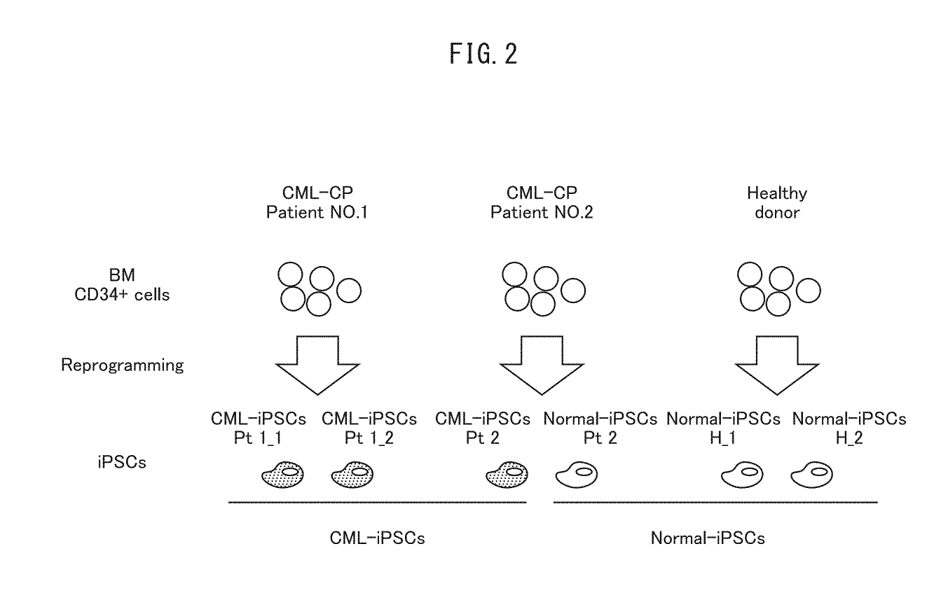

The method described in Kumano, K. et al., Blood 119, 6234-42 (2012) was used to prepare iPSC from CML-CP patient derived cells. CD34+ cells purified from bone marrow samples from two patients newly diagnosed with CML-CP were grown for 2 days in α-MEM medium supplemented with 20% fetal bovine serum (FCS), stem-cell factor (SCF), Fms-like tyrosine kinase-3 ligand (Flt3L), interleukin (IL)-3, IL-6 and thrombopoietin (TPO), as described in the literature. A plasmid mixture containing pCXLE-hOCT3/4-shp53-F, pCXLE-hSK, pCXLE-hUL and pCXWB-EBNA1 was used for electroporation in 2×10⁵ CD34+ cells, as described in the literature. The CD34+ cells were then cultured for 20-30 days with mouse embryo fibroblasts to obtain CML-CP patient-derived embryonic stem cell (ES)-like colonies. Expression of stem cell genes and BCR-ABL were confirmed by RT-PCR (FIG. 1). As an exception, the iPSCs (CML Pt2 iPSCs No. 3) from one of the patients did not express BCR-ABL, and were normal iPSCs. The CML-CP patient-derived iPSCs expressing BCR-ABL (CML Pt1 iPSCs No. 3 and 5 and CML Pt2 iPSCs No. 4 in FIG. 1) will hereunder be referred to as “CML-iPSCs”. Cells from a healthy human were also reprogrammed, to prepare iPSCs using the same method as above. These obtained iPSCs will hereunder be referred to as “normal iPSCs”. The scheme for the experiment is shown in FIG. 2.

(2) Hematopoietic Differentiation of iPSCs

Hematopoietic differentiation of the CML-iPSCs was carried out according to Takayama, N. et al., J. Exp. Med. 207, 2817-30 (2010). The iPSC clusters were transferred onto C3H10T1/2 cells treated with mitomycin C, and co-cultured in hematopoietic differentiation medium in the presence of 20 ng/ml human vascular endothelial growth factor (VEGF). The medium consisted of Iscove's modified Dulbecco's medium containing a cocktail of 10 mg/ml human insulin, 5.5 mg/ml human transferrin, 5 ng/ml sodium selenite, 2 mmol/L L-glutamine, 0.45 mmol/L monothioglycerol, 50 mg/ml ascorbic acid and 15% highly filtered fetal bovine serum (FBS). The medium was replaced on day 4, 7, 10, 12, 14 and 16 from the start of culturing. After 17-18 days of culturing, the iPSC sacs crushed with pipette tips were filtered with a cell strainer. CD34+/CD45− pre-hematopoietic progenitor cells (hereunder also referred to as “CML-pre-HPC”) and CD34−/CD45+ differentiated cells (hereunder also referred to as “CML-DC”) were isolated from CD43+ hematopoietic cells (HC) by flow cytometry. In the same manner, differentiation of normal iPSCs was induced to prepare pre-HPCs and DCs (hereunder also referred to as “normal-pre-HPC” and “normal-DC”, respectively).

### (3) Cell Proliferation Assay

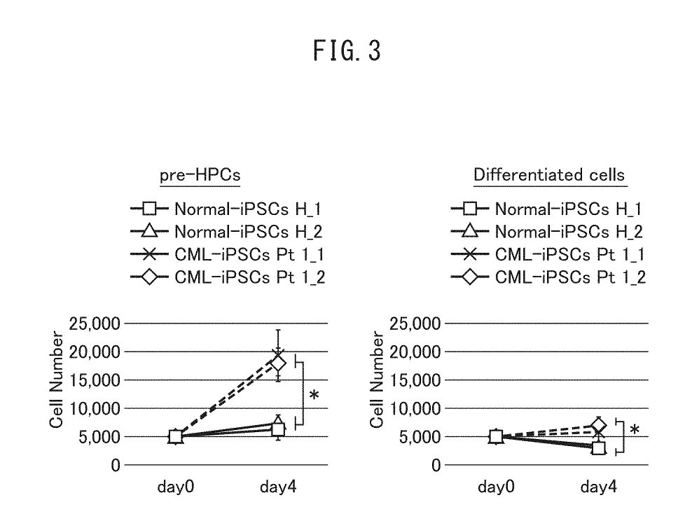

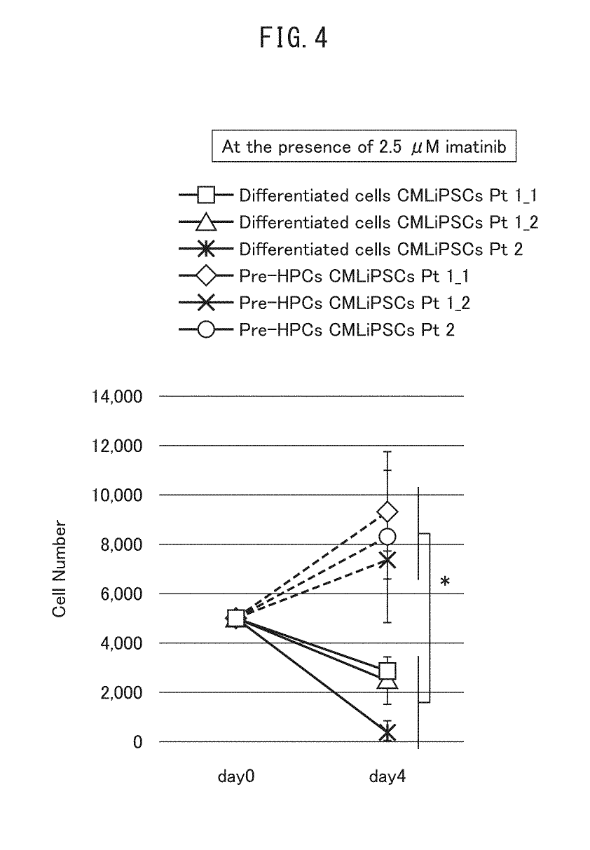

A cell proliferation assay was performed according to the method described in Kumano, K. et al., Blood 119, 6234-42 (2012). In the presence or in the absence of imatinib (2.5 M) as a tyrosine kinase inhibitor, 5,000 iPSC-derived pre-HPCs and DCs were cultured in α-MEM containing 20% FCS supplemented with 100 ng/ml SCF, 10 ng/ml TPO, 100 ng/ml Flt3L, 10 ng/ml IL3 and 100 ng/ml IL6. Each assay was performed in duplicate. The results in the absence and in the presence of imatinib are shown in FIGS. 3 and 4, respectively.

CML-pre-HPCs exhibited high cell proliferation compared to the normal-pre-HPCs (FIG. 3). Moreover, the CML-pre-HPCs also continued to proliferate even in the presence of imatinib. However, the CML-DC cell count was reduced in the presence of imatinib (FIG. 4).

### (4) Apoptosis Assay

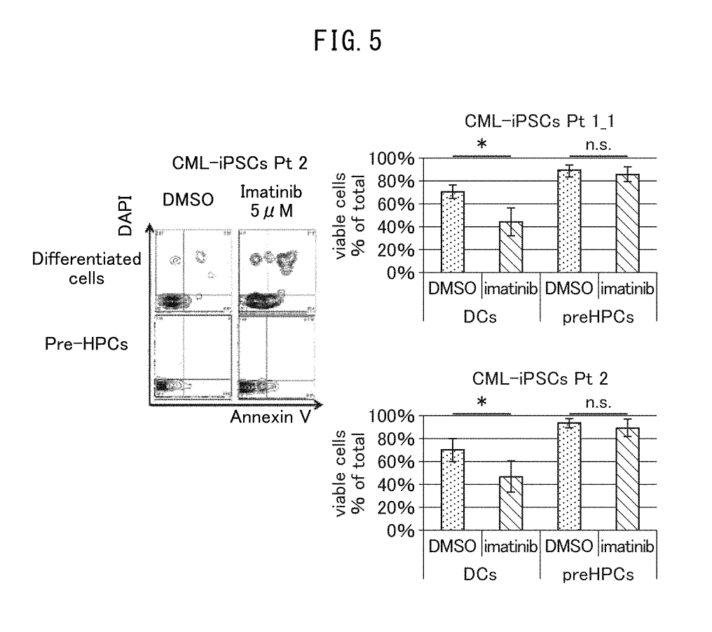

In the hematopoietic differentiation step of (2) above, an apoptosis assay was carried out by medium exchange with medium containing or not containing 5 μM imatinib, on the 16th day from the start of the process. The results after 48 hours from medium exchange are shown in FIG. 5. The CML-pre-HPCs were not induced to undergo apoptosis even when imatinib was added. The results of (3) and (4) suggest that the CML-pre-HPCs not only reproduced the phenotype of CML, but also had resistance to imatinib, which is the major feature of CML stem cells.

### (5) Microarray Analysis

The gene expression analysis was performed using SurePrint G3 Human GE 8x60K v2 Microarray (Agilent). Published gene expression microarray data was also analyzed, based on a human sample from the Gene Expression Omnibus (GEO) database (GEO GSE40721). Standardization of the raw data and clustering analysis were performed using CLC Genomics Workbench. For comparison of the pre-HPC and DC expression profiles, standardized data were tested in relation to Gene Set Enrichment Analysis (GSEA) using selected gene sets. In order to screen for genes having high expression levels in the CML-pre-HPCs compared to the CML-DCs, individual gene expressions were compared among groups. Genes were selected that had increased expression in CML-pre-HPCs compared to CML-DCs, based on the unpaired Student's t-test (P<0.05).

The following 23 gene sets were concentrated from 3267 selected gene sets, by GSEA for the CML-pre-HPCs compared to the CML-DCs, under conditions without imatinib (false detection rate, q value <0.05). They include a gene upregulated by p53 inactivation and the target gene of NUP98-HOXA9. Both genes are known to contribute to TKI resistance in CML.

TABLE 6

| Gene set | | FDR |
|---|---|---|
| No. | Gene set name | q-value |
| 1 | REACTOME_ABACAVIR_TRANSPORT_AND_METABOLISM | 0.041 |
| 2 | TANG_SENESCENCE_TP53_TARGETS_UP | 0.041 |
| 3 | BIOCARTA_NEUROTRANSMITIERS_PATHWAY | 0.041 |
| 4 | WANG_RESPONSE_TO_PACLITAXEL_VIA_MAPK8_UP | 0.041 |
| 5 | NAKAJIMA_MAST_CELL | 0.041 |
| 6 | VERHAAK_AML_WITH_NPM1_MUTATED_DN | 0.041 |
| 7 | WILLIAMS_ESR2_TARGETS_UP | 0.041 |
| 8 | REACTOME_NITRIC_OXIDE_STIMULATES_GUANYLATE_CYCLASE | 0.041 |
| 9 | TAKEDA_TARGETS_OF_NUP98_HOXA9_FUSION_8D_UP | 0.041 |
| 10 | YAGUE_PRETUMOR_DRUG_RESISTANCE_UP | 0.041 |
| 11 | SUMI_HNF4A_TARGETS | 0.041 |
| 12 | LOPEZ_EPITHELIOID_MESOTHELIOMA | 0.041 |
| 13 | HOEGERKORP_CD44_TARGETS_TEMPORAL_DN | 0.045 |
| 14 | PEPPER_CHRONIC_LYMPHOCYTIC_LEUKEMIA_DN | 0.045 |
| 15 | TAKEDA_TARGETS_OF_NUP98_HOXA9_FUSION_I6D_UP | 0.045 |
| 16 | REACTOME_GLUCAGON_SIGNALING_IN_METABOLIC_REGULATION | 0.044 |
| 17 | TAKEDA_TARGETS_OF_NUP98_HOXA9_FUSION_3D_UP | 0.044 |
| 18 | WINTER_HYPOXIA_DN | 0.046 |
| 19 | WAMUNYOKOLI_OVARIAN_CANCER_GRADES_1_2_DN | 0.046 |
| 20 | NEWMAN_ERCC6_TARGETS_UP | 0.046 |
| 21 | CHEN_NEUROBLASTOMA_COPY_NUMBER_GAINS | 0.046 |
| 22 | WIERRENGA_STAT5A_TARGETS_DN | 0.048 |
| 23 | OUILLETTE_CLL_13Q14_DELETION_UP | 0.048 |

### (6) Evaluation of TKI Resistance in CML-Pre-HPCs

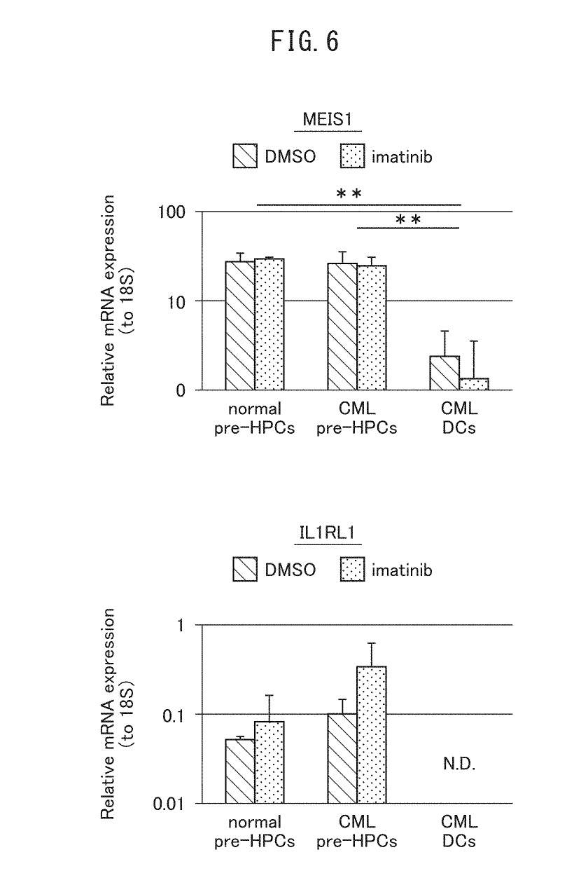

As candidate genes contributing to imatinib resistance there were selected 166 genes with increased expression levels in CML-pre-HPCs by imatinib treatment. Of the 166 genes, IK1RL1, which has been reported as a gene contributing to imatinib resistance, exhibited the highest expression level. The expression levels of both IK1RL1 and MEIS1, the target of NUP98-HOXA9, were evaluated by real-time quantitative RT-PCR. The results are shown in FIG. 6. The results indicated that CML-pre-HPCs can be used as a TKI-resistant CML stem cell model.

### Example 2

### Identification of ADAM8 as a Marker for TKI-Resistant CML Cells

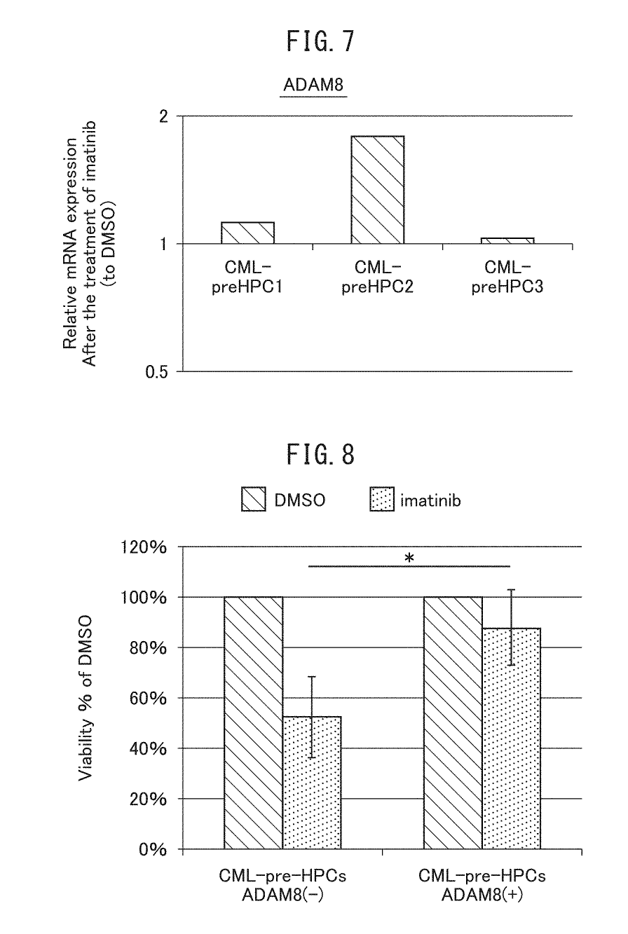

Based on analysis relating to the CML stem cells among the published array data for CML patients (GSE40721), 6 genes (CYTH4, PTMA, OLFM2, ADAM8, POGK, PCKDK) were selected from among the 166 candidate genes. The 6 genes had higher expression levels in the TKI-sensitive CML stem cell fraction (CD34+/CD38−) than in the TKI-resistant CML progenitor cell fraction (CD34+/CD38+). The expression levels of the 6 genes were confirmed by real-time quantitative RT-PCR. FIG. 7 shows the evaluation results for the ADAM8 RNA expression level, as one of the 6 genes. As a result of the cell viability assay, the ADAM8-positive CML-pre-HPC cells exhibited greater TKI resistance compared to the ADAM8-negative CML-pre-HPC cells (FIG. 8). These results indicate that ADAM8 is an excellent biomarker for TKI-resistant CML cells.

### Example 3

Test Using Primary Samples from Patients Newly Diagnosed as CML-CP

### (1) Evaluation of CD34+ADAM8+ Cell Fraction

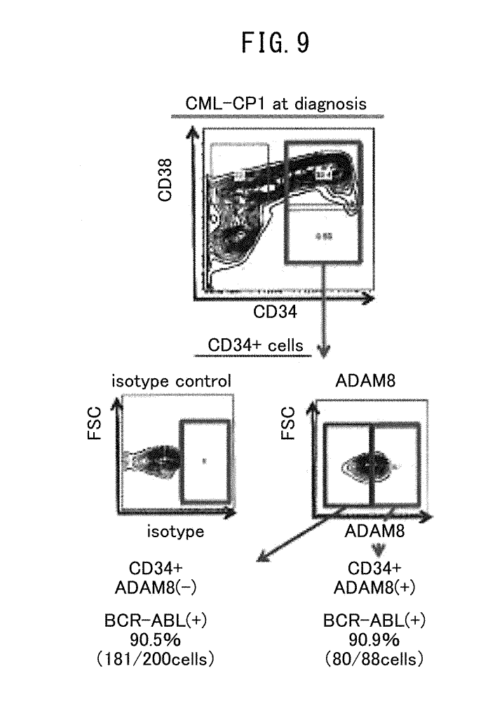

In order to confirm that ADAM8 functions as a TKI-resistant CML cell marker in CML-CP patients, a CD34+ADAM8+ cell fraction purified by Fluorescence Activated Cell Sorting (FACS) from a primary sample from a patient newly diagnosed as CML-CP was evaluated (FIG. 9).

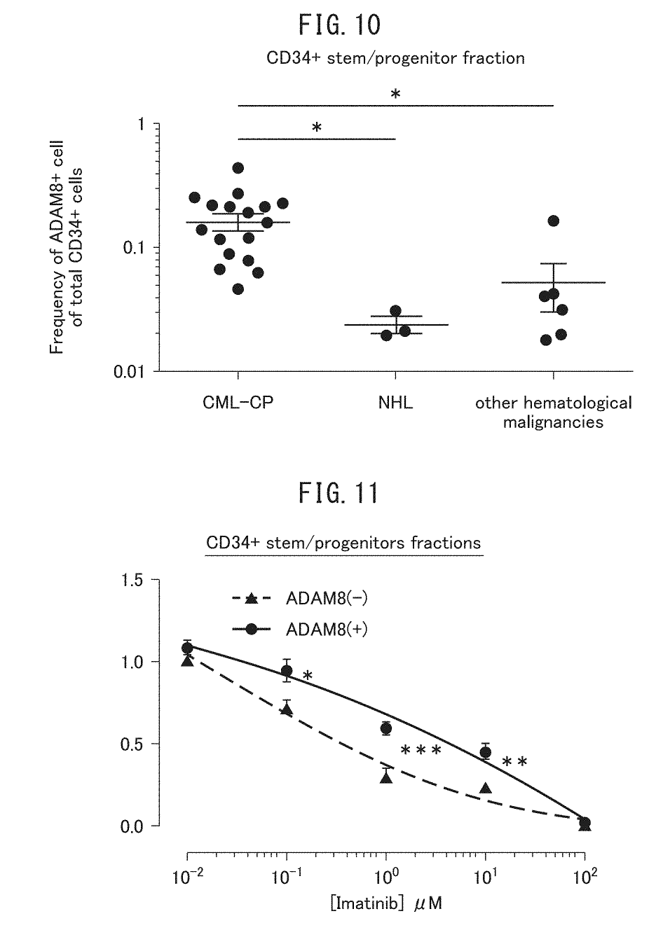

Based on FCM analysis, the CD34+ stem cell/progenitor cell fraction in the CML-CP patient bone marrow sample was shown to have more abundant ADAM8+ cells compared to the CD34+ stem cell/progenitor cell fraction in the sample from an NHL (normal control) patient and the CD34+ stem cell/progenitor cell fraction in the sample from another malignant blood disease patient (FIG. 10).

### (2) Cell Viability Assay and Apoptosis Assay of CD34+ADAM8+ Cell Fraction and CD34+ADAM8− Cell Fraction.

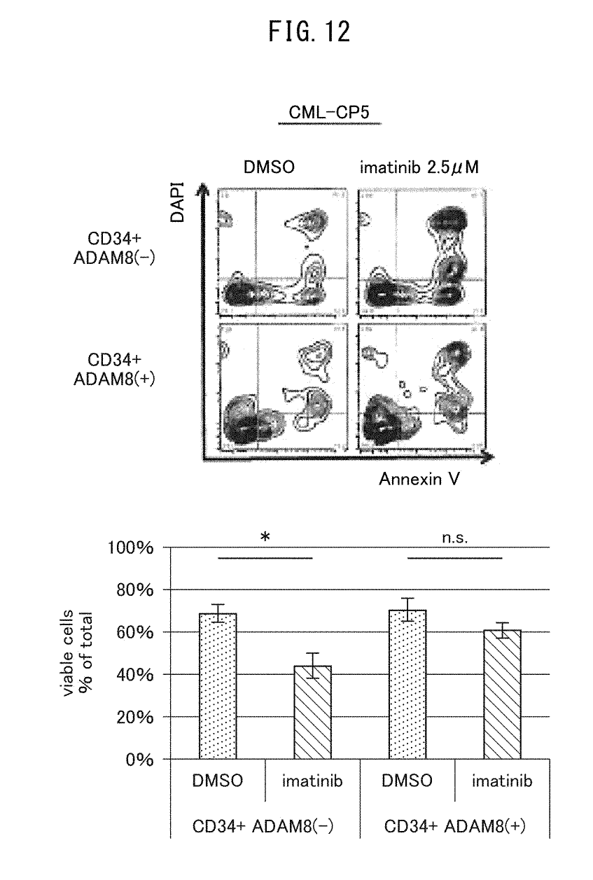

The cell viability assay and apoptosis assay were carried out according to the method described in Ma, L. et al., Sci. Transl. Med. 6, 252ral21(2014). The purified CML-CP sample was cultured in α-MEM containing 20% FCS supplemented with 100 ng/ml SCF, 100 ng/ml granulocyte colony stimulating factor (G-CSF), 20 ng/ml FL3L, 20 ng/ml IL3 and 20 ng/ml IL6, in the presence and in the absence of imatinib. After 48 hours from exposure to imatinib, the cells were stained with annexin-V and 4,6-diamidino-2-phenylindole antibody, according to the manufacturer's protocol. The results are shown in FIGS. 11 and 12.

The ADAM8+CML cells of the CML-CP patient exhibited greater imatinib resistance compared to ADAM8−CML cells (FIG. 11). Moreover, apoptosis induced by imatinib was suppressed in ADAM8+CML cells compared to ADAM8-CML cells (FIG. 12).

### (3) Evaluation of CD34+CD38+ADAM8+ Cell Fraction and CD34+CD38−ADAM8+ Cell Fraction

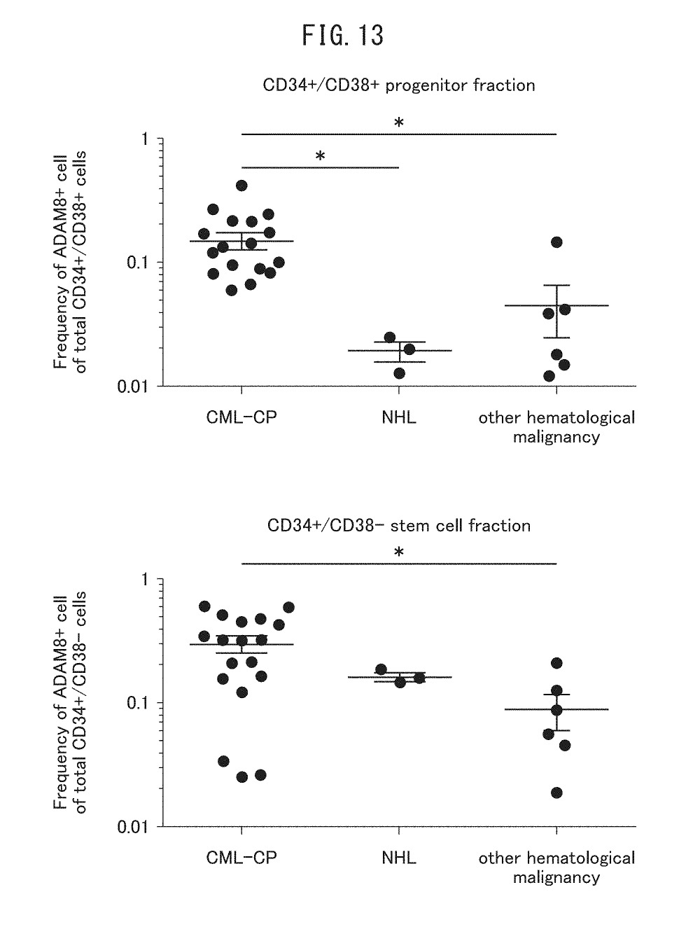

The frequencies of ADAM8+ cells in the CD34+CD38+ cell fraction (progenitor fraction) and CD34+CD38− cell fraction (stem cell fraction) were measured. The results are shown in FIG. 13. In the CD34+CD38+ progenitor fraction, the frequency of ADAM8+ cells was significantly higher in CML-CP patients (n=18) than in NHL patients (n=3) and other blood malignant tumor patients (AML: n=1, ALL: n=1, MDS: n=1, CMML: n=3).

### (4) Cell Viability Assay of CD34+CD38+ADAM8+ Cell Fraction and CD34+CD38+ADAM8− Cell Fraction

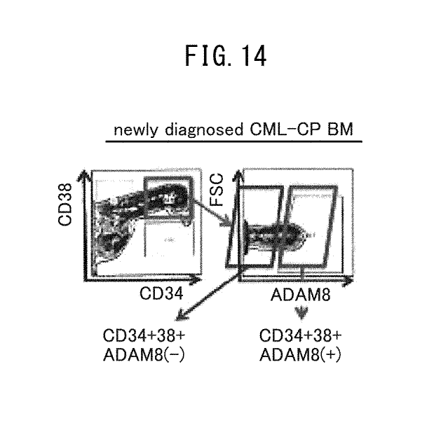

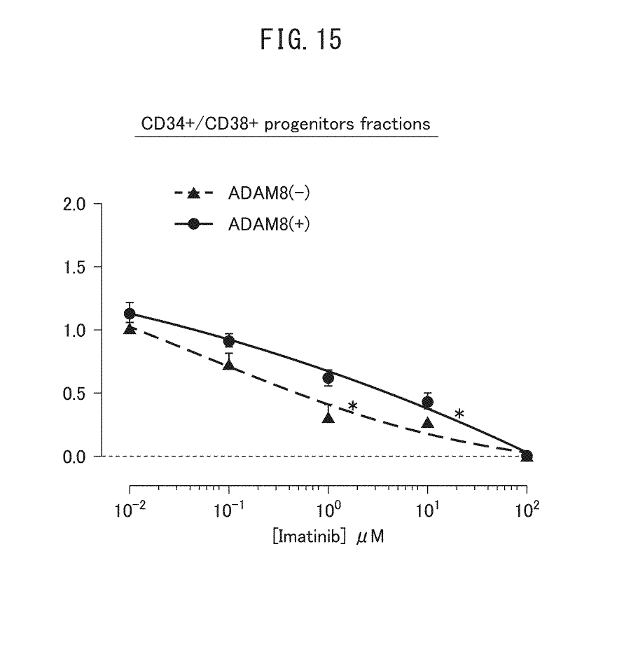

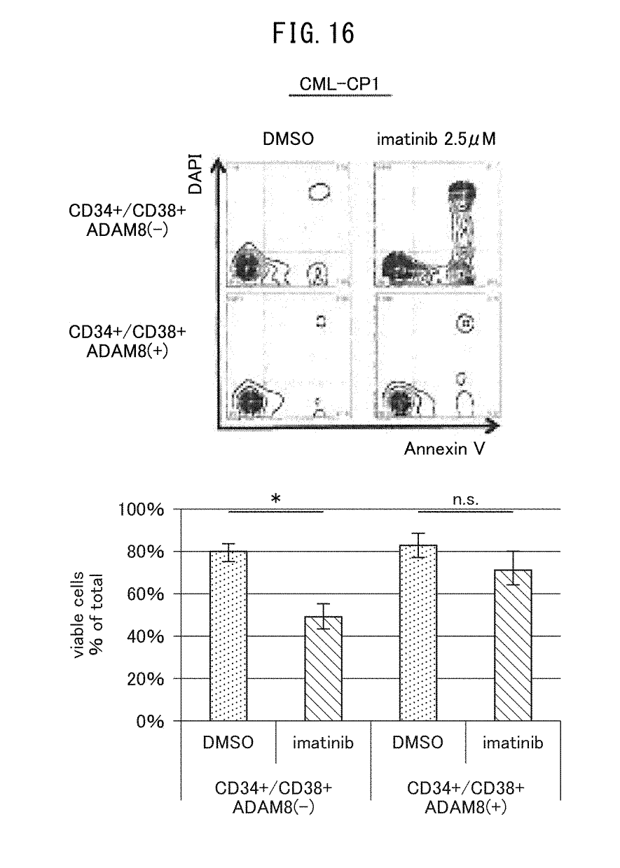

The ADAM8+ cell fraction and ADAM8− cell fraction were separated from the CD34+CD38+ cell fraction (progenitor cell fraction) by FACS (FIG. 14). The CD34+CD38+ADAM8+ cell fraction and the CD34+CD38+ADAM8− cell fraction were used for a cell viability assay and apoptosis assay in the same manner as (2) above. The results are shown in FIG. 15 and FIG. 16.

The ADAM8+ cells exhibited greater TKI resistance than the ADAM8− cells, even for CD34+/CD38+ progenitor cells which are known as being TKI-sensitive. This indicates that ADAM8 is a useful biomarker for TKI-resistant CML cells.

### Example 4

Test Using Samples from Patients Reaching MMR and MUL by TKI Treatment

Samples obtained from patients that had reached MMR (major molecular response) (n=2) and MUL (molecular undetectable leukemia) (n=1) by TKI treatment were evaluated by a limiting dilution assay.

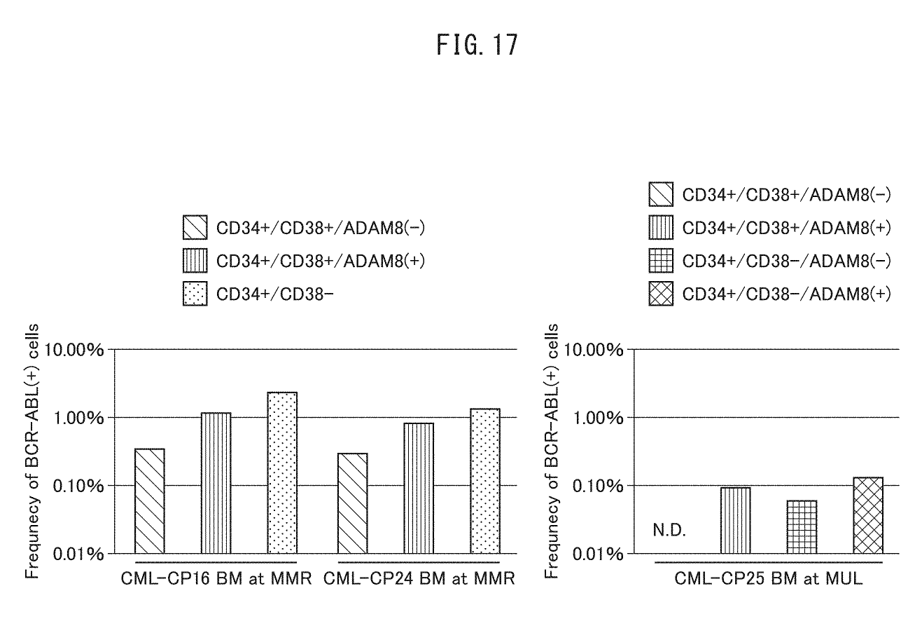

Eight dilution series were prepared from the CD34+CD38+ADAM8+ cell fraction separated by FACS, at 300 cells/well, 100 cells/well, 33 cells/well and 11 cells/well of a 96-well plate. Eight dilution series were also prepared from the CD34+CD38+ADAM8− cell fraction, at 900 cells/well, 300 cells/well, 100 cells/well and 33 cells/well of a 96-well plate. For each well, one-step RT-PCR was carried out for BCR-ABL by the method described in Chu, S. et al., Blood. 2011 Nov. 17; 118(20):5565-72, and the absolute frequency of BCR-ABL-expressing cells was calculated according to Hu, Y et al., J Immunol Methods. 347, 70-8 (2009). The results are shown in FIG. 17.

For the MMR CML patients, the frequency of BCR-ABL+ cells was higher in the CD34+/CD38+/ADAM8+ cell fraction than in the CD34+/CD38+/ADAM8− cell fraction. The frequency of BCR-ABL+ cells was about the same in the CD34+/CD38+/ADAM8+ cell fraction and the CD34+/CD38− cell fraction. For the MUL patients, BCR-ABL+ cells were detected in the ADAM8+ cell fraction. However, BCR-ABL+ cells were not detected in the CD34+/CD38+/ADAM8− cell fraction. These findings suggest that ADAM8 can be used as a biomarker for residual TKI-resistant CML cells present in a CML-CP patient that exhibits a satisfactory response against TKI.

### Example 5

### Effect of ADAM8 Gene Expression Inhibition

(1) Evaluation of ADAM8 Gene Expression Inhibition by shRNA

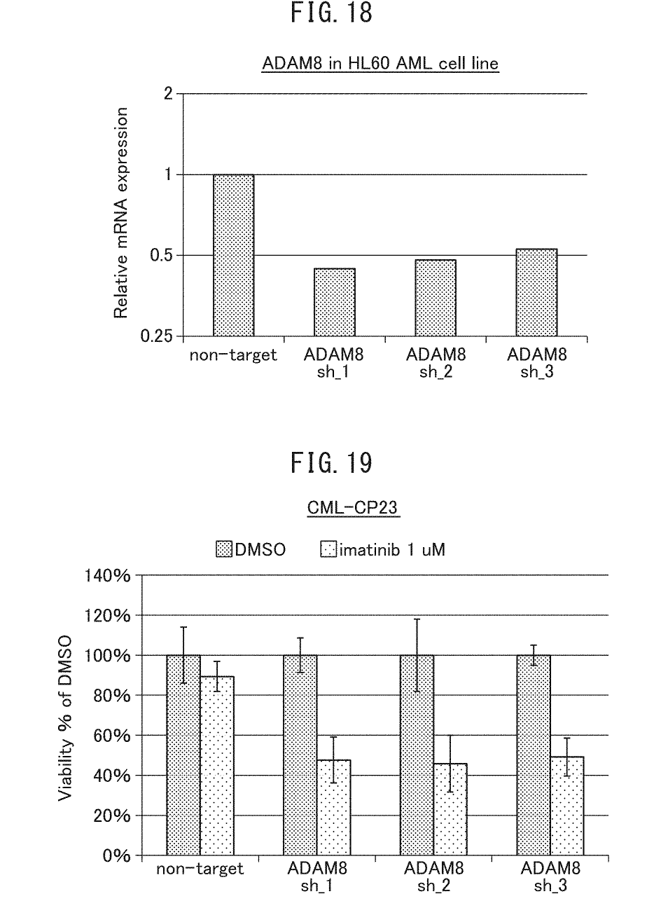

The amount of mRNA transcribed from the ADAM8 gene in the cells was measured after treatment of an HL60AML cell line with ADAM8_shRNA 1 (SEQ ID NO: 45), ADAM8_shRNA 2 (SEQ ID NO: 46) and ADAM8_shRNA_3 (SEQ ID NO: 47). The target sequences of ADAM8_shRNA_1, ADAM8_shRNA_2 and ADAM8_shRNA_3 are listed as SEQ ID NO: 49, 50 and 51, respectively. Specifically, pLKO.1-puro-CMV-tGFP (Sigma Aldrich Japan) incorporating DNA encoding shRNA was transferred into HEK293 cells together with pMISSION GAG POL (Sigma Aldrich Japan) and pMISSION VSV-G (Sigma Aldrich Japan), to obtain an shRNA-expressing lentivirus. The obtained lentivirus was used to infect HL60, 1 μg/ml puromycin was used for 72 hours of selection, and ADAM8 gene expression in the selected cells was analyzed. The results are shown in FIG. 18. The expression level of the ADAM8 gene when using non-target_shRNA that targets a non-ADAM8 gene (SEQ ID NO: 48, target sequence: SEQ ID NO: 52) (indicated as “non-target” in the graph) was defined as 1. ADAM8_shRNA_1 to 3 were confirmed to have the ability to knockdown ADAM8.

### (2) Cell Viability Assay Using CD34+ADAM8+ Cells

The shRNA-expressing lentivirus obtained in (1) above was used to infect CML-CP23-derived CD34+ADAM8+ cells. Puromycin-resistant cells were selected by 60 hours of treatment with 2.5 μg/ml puromycin. The obtained cells were subjected to a cell viability assay in the absence of imatinib and in the presence of 1.0 μM imatinib, in the same manner as Example 3(2). The results are shown in FIG. 19. This demonstrated that TKI resistance is lowered by inhibition of ADAM8 gene expression.

### Example 6

### Effect of ADAM8 Activity Inhibition

### (1) Evaluation of Effect of ADAM8 Activity Inhibitors on CD34+ADAM8+ Cells and CD34+ADAM8− Cells

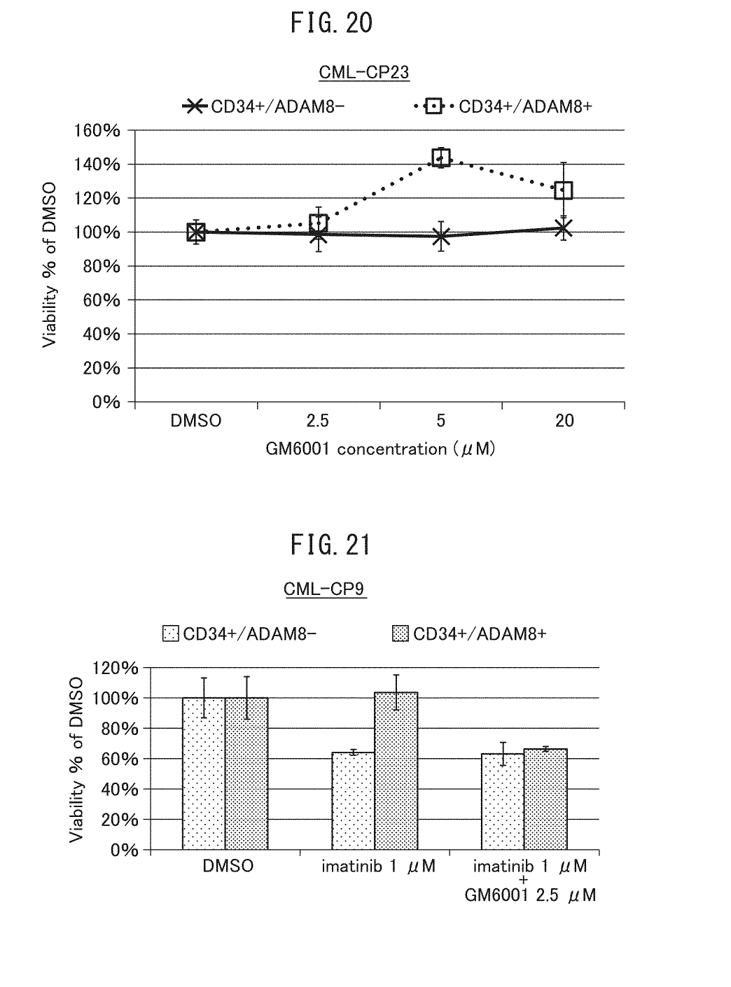

CD34+ADAM8+ cells and CD34+ADAM8− cells were used for a cell viability assay in the absence of GM6001 (Merck Millipore) and in the presence of GM6001 at specific concentrations (2.5 μM, 5 μM, 20 μM). The cell viability assay was carried out by the same method as Example 3(2), except for using GM6001 instead of imatinib. The results are shown in FIG. 20. GM6001 was confirmed to have no effect on the cell viability of either CD34+ADAM8+ cells or CD34+ADAM8− cells, at a concentration of 2.5 μM.

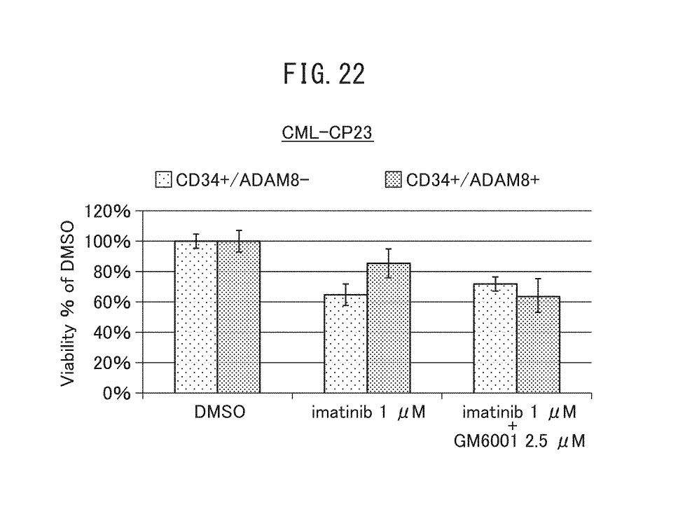

CD34+ADAM8+ cells and CD34+ADAM8− cells were used for a cell viability assay in the absence of imatinib and GM6001, in the presence of 1.0 μM imatinib, or in the presence of 1.0 μM imatinib and 2.5 μM GM6001. The cell viability assay in the absence of imatinib and GM6001 and in the presence of 1.0 μM imatinib was carried out by the same method as Example 3(2). The cell viability assay in the presence of 1.0 μM imatinib and 2.5 μM GM6001 was carried out by the same method as Example 3(2), except for using GM6001 in addition to imatinib. The results are shown in FIG. 21 and FIG. 22. This demonstrated that TKI resistance is lowered by inhibition of ADAM8 activity.

### INDUSTRIAL APPLICABILITY

The present invention can be suitably utilized for detection of tyrosine kinase inhibitor-resistant chronic myelogenous leukemia cells with high sensitivity, and for their isolation. The present invention also makes it possible to efficiently carry out diagnosis of chronic myelogenous leukemia, monitoring of chronic myelogenous leukemia, classification of patient groups in need of treatment for chronic myelogenous leukemia, and prediction of relapse of chronic myelogenous leukemia. Moreover, according to the invention it is possible to lower TKI resistance in CML.

### SEQUENCE LISTING

1. A method for testing chronic myelogenous leukemia, comprising a step of detecting expression of the ADAM8 gene in blood cells in a sample obtained from a subject.

2. The method according to claim 1, wherein the subject is a subject undergoing treatment with a tyrosine kinase inhibitor.

3. The method according to claim 1, wherein the subject is a subject that has reached MMR (major molecular response) or MUL (molecular undetectable leukemia) by treatment using a tyrosine kinase inhibitor.

4. The method according to claim 1, which is a method that aids in classification of a patient in need of treatment for chronic myelogenous leukemia.

5. The method according to claim 1, which is a method that aids in predicting relapse of chronic myelogenous leukemia.

6. The method according to claim 2, wherein the tyrosine kinase inhibitor is selected from the group consisting of imatinib, gefitinib, erlotinib, osimertinib, afatinib, dasatinib, bosutinib, vandetanib, sunitinib, axitinib, pazopanib, lenvatinib, lapatinib, nintedanib, nilotinib, ibrutinib and ponatinib.

7. The method according to claim 1, wherein the sample is peripheral blood or bone marrow fluid.

8. The method according to claim 1, wherein the blood cells are CD34-positive cells.

9. The method according to claim 1, wherein detecting expression of the ADAM8 gene includes measurement of the number of cells expressing the ADAM8 gene.

10. The method according to claim 1, wherein detecting expression of the ADAM8 gene includes measurement of the amount of mRNA transcribed from the ADAM8 gene.

11. The method according to claim 1, wherein detecting expression of the ADAM8 gene includes measurement of the amount of protein encoded by the ADAM8 gene.

12. A method for isolating chronic myelogenous leukemia cells having resistance to a tyrosine kinase inhibitor from blood cells in a sample obtained from a subject, the method comprising steps of:
(1) detecting expression of the ADAM8 gene in blood cells in a sample; and
(2) isolating chronic myelogenous leukemia cells having resistance to the tyrosine kinase inhibitor, based on the detection results of step (1).

13. A kit for testing chronic myelogenous leukemia to be used in the method according to claim 1, comprising a detection reagent for mRNA transcribed from the ADAM8 gene or a detection reagent for a protein encoded by the ADAM8 gene.


SEQUENCE LISTING (52 sequences)

SEQ ID NO: 1 - RNA, 3316 nt, Homo sapiens
gcggaaaagagccucgggccaggagcgcaggaaccagaccgugucccgcggggcugucaccuccgccucugcuccccgacccggccaugcgcggccucgggcucuggcugcugggcgcgaugaugcugccugcgauugcccccagccggcccugggcccucauggagcaguaugaggucguguugccguggcgucugccaggcccccgaguccgccgagcucugcccucccacuugggccugcacccagagagggugagcuacguccuuggggccacagggcacaacuucacccuccaccugcggaagaacagggaccugcugggcuccggcuacacagagaccuauacggcugccaauggcuccgaggugacggagcagccucgcgggcaggaccacugcuucuaccagggccacguagagggguacccggacucagccgccagccucagcaccugugccggccucagggguuucuuccagguggggucagaccugcaccugaucgagccccuggaugaagguggcgagggcggacggcacgccguguaccaggcugagcaccugcugcagacggccgggaccugcggggucagcgacgacagccugggcagccuccugggaccccggacggcagccgucuucaggccucggcccggggacucucugccaucccgagagacccgcuacguggagcuguauguggucguggacaaugcagaguuccagaugcuggggagcgaagcagccgugcgucaucgggugcuggagguggugaaucacguggacaagcuauaucagaaacucaacuuccgugugguccuggugggccuggagauuuggaauagucaggacagguuccacgucagccccgaccccagugucacacuggagaaccuccugaccuggcaggcacggcaacggacacggcggcaccugcaugacaacguacagcucaucacgggugucgacuucaccgggacuaccgugggguuugccaggguguccgccaugugcucccacagcucaggggcugugaaccaggaccacagcaagaaccccgugggcguggccuguaccauggcccaugagaugggccacaaccugggcauggaccaugaugagaacguccagggcugccgcugccaggaacgcuucgaggccggccgcugcaucauggcgggcagcauuggcuccaguuuccccaggauguucagugacugcagccaggccuaccuggagagcuuuuuggagcggccgcagucggugugccucgccaacgccccugaccucagccaccuggugggcggccccgugugugggaaccuguuuguggagcguggggagcagugcgacugcggcccccccgaggacugccggaaccgcugcugcaacucuaccaccugccagcuggcugagggggcccagugugcgcacgguaccugcugccaggagugcaaggugaagccggcuggugagcugugccgucccaagaaggacaugugugaccucgaggaguucugugacggccggcacccugagugcccggaagacgccuuccaggagaacggcacgcccugcuccgggggcuacugcuacaacggggccugucccacacuggcccagcagugccaggccuucugggggccaggugggcaggcugccgaggaguccugcuucuccuaugacauccuaccaggcugcaaggccagccgguacagggcugacauguguggcguucugcagugcaagggugggcagcagccccuggggcgugccaucugcaucguggaugugugccacgcgcucaccacagaggauggcacugcguaugaaccagugcccgagggcacccgguguggaccagagaagguuugcuggaaaggacguugccaggacuuacacguuuacagauccagcaacugcucugcccagugccacaaccauggggugugcaaccacaagcaggagugccacugccacgcgggcugggccccgccccacugcgcgaagcugcugacugaggugcacgcagcguccgggagccuccccgucuucgugguggugguucuggugcuccuggcaguugugcuggucacccuggcaggcaucaucgucuaccgcaaagcccggagccgcauccugagcaggaacguggcucccaagaccacaauggggcgcuccaacccccuguuccaccaggcugccagccgcgugccggccaagggcggggcuccagccccauccaggggcccccaagagcugguccccaccacccacccgggccagcccgcccgacacccggccuccucgguggcucugaagaggccgcccccugcuccuccggucacuguguccagcccacccuucccaguuccugucuacacccggcaggcaccaaagcaggucaucaagccaacguucgcacccccagugcccccagucaaacccggggcuggugcggccaacccugguccagcugagggugcuguuggcccaaagguugcccugaagccccccauccagaggaagcaaggagccggagcucccacagcacccuaggggggcaccugcgccuguguggaaauuuggagaaguugcggcagagaagccaugcguuccagcauuccacgguccagcuagugccgcucagcccuagacccugacuuugcaggcucagcugcuguucuaaccucaggaaugcaucuaccugagaggcuccugcuguccacgcccucagccaauuccuucuccccgccuuggccacguguagccccagcugucugcaggcaccaggcugggaugagcugugugcuugcgggugcguguguguguacgugucuccagguggccgcuggucucccgcuguguucaggaggccacauauacagccccucccagccacaccugccccugcucuggggccugcugagccggcugcccugggcacccgguuccaggcagcacagacguggggcauccccagaaagacuccaucccaggaccagguuccccugcgugcucuucgagagggugucagugagcagacugcaccccaagcucccgacuccagguccccugaucuuggggccuguuucccaugggauucaagagggacagccccagcuuuguguguguuuaagcuuaggaaucgccuuuauggaaagggcuaugugggagagucagcuaucuugucugguuuucuugagaccucagauguguguucagcagggcugaaagcuuuuauucuuuaauaaugagaaauguauauuuuacuaauaaauuauugaccgaguucuguaaaaaaaaaaaaaaaa

SEQ ID NO: 2 - protein, 824 aa, Homo sapiens
MRGLGLWLLGAMMLPAIAPSRPWALMEQYEVVLPWRLPGPRVRRALPSHLGLHPERVSYVLGATGHNFTLHLRKNRDLLGSGYTETYTAANGSEVTEQPRGQDHCFYQGHVEGYPDSAASLSTCAGLRGFFQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCWKGRCQDLHVYRSSNCSAQCHNHGVCNHKQECHCHAGWAPPHCAKLLTEVHAASGSLPVFVVVVLVLLAVVLVTLAGIIVYRKARSRILSRNVAPKTTMGRSNPLFHQAASRVPAKGGAPAPSRGPQELVPTTHPGQPARHPASSVALKRPPPAPPVTVSSPPFPVPVYTRQAPKQVIKPTFAPPVPPVKPGAGAANPGPAEGAVGPKVALKPPIQRKQGAGAPTAP

SEQ ID NO: 3 - DNA, 21 bp, Artificial Sequence
  primer
cgatgatgctgcctgcgattg

SEQ ID NO: 4 - DNA, 20 bp, Artificial Sequence
  primer
cgcaggtggagggtgaagtt

SEQ ID NO: 5 - DNA, 23 bp, Artificial Sequence
  primer
atggggttttggatcttagcaat

SEQ ID NO: 6 - DNA, 23 bp, Artificial Sequence
  primer
cacggtgtaactaggttttcctt

SEQ ID NO: 7 - DNA, 21 bp, Artificial Sequence
  primer
gatatagccgtgttcgccaaa

SEQ ID NO: 8 - DNA, 21 bp, Artificial Sequence
  primer
cggtggcagaaattgtcacat

SEQ ID NO: 9 - DNA, 21 bp, Artificial Sequence
  primer
aactccagactgtccacagca

SEQ ID NO: 10 - DNA, 18 bp, Artificial Sequence
  primer
aacgagcggcttcactca

SEQ ID NO: 11 - DNA, 20 bp, Artificial Sequence
  primer
gtaacccgttgaaccccatt

SEQ ID NO: 12 - DNA, 20 bp, Artificial Sequence
  primer
ccatccaatcggtagtagcg

SEQ ID NO: 13 - DNA, 23 bp, Artificial Sequence
  primer
ccccagggccccattttggtacc

SEQ ID NO: 14 - DNA, 26 bp, Artificial Sequence
  primer
acctcagtttgaatgcatgggagagc

SEQ ID NO: 15 - DNA, 20 bp, Artificial Sequence
  primer
cattcaaactgaggtaaggg

SEQ ID NO: 16 - DNA, 22 bp, Artificial Sequence
  primer
tagcgtaaaaggagcaacatag

SEQ ID NO: 17 - DNA, 24 bp, Artificial Sequence
  primer
acccatccttcctgcccgatcaga

SEQ ID NO: 18 - DNA, 24 bp, Artificial Sequence
  primer
ttggtaatggagcggcgggacttg

SEQ ID NO: 19 - DNA, 23 bp, Artificial Sequence
  primer
ccacctcgccttacacatgaaga

SEQ ID NO: 20 - DNA, 22 bp, Artificial Sequence
  primer
tagcgtaaaaggagcaacatag

SEQ ID NO: 21 - DNA, 24 bp, Artificial Sequence
  primer
ttcacatgtcccagcactaccaga

SEQ ID NO: 22 - DNA, 26 bp, Artificial Sequence
  primer
tcacatgtgtgagaggggcagtgtgc

SEQ ID NO: 23 - DNA, 24 bp, Artificial Sequence
  primer
ttcacatgtcccagcactaccaga

SEQ ID NO: 24 - DNA, 22 bp, Artificial Sequence
  primer
tttgtttgacaggagcgacaat

SEQ ID NO: 25 - DNA, 26 bp, Artificial Sequence
  primer
gcgaacccaagacccaggcctgctcc

SEQ ID NO: 26 - DNA, 25 bp, Artificial Sequence
  primer
cagggggtctgctcgcaccgtgatg

SEQ ID NO: 27 - DNA, 21 bp, Artificial Sequence
  primer
ggctgagaagaggatggctac

SEQ ID NO: 28 - DNA, 22 bp, Artificial Sequence
  primer
tttgtttgacaggagcgacaat

SEQ ID NO: 29 - DNA, 25 bp, Artificial Sequence
  primer
agccatatggtagcctcatgtccgc

SEQ ID NO: 30 - DNA, 30 bp, Artificial Sequence
  primer
tcaattctgtgcctccgggagcagggtagg

SEQ ID NO: 31 - DNA, 25 bp, Artificial Sequence
  primer
agccatatggtagcctcatgtccgc

SEQ ID NO: 32 - DNA, 22 bp, Artificial Sequence
  primer
tagcgtaaaaggagcaacatag

SEQ ID NO: 33 - DNA, 23 bp, Artificial Sequence
  primer
atcagggccaagacatagagatg

SEQ ID NO: 34 - DNA, 20 bp, Artificial Sequence
  primer
gccaatgcaacttggacgtt

SEQ ID NO: 35 - DNA, 20 bp, Artificial Sequence
  primer
accacagtccatgccatcac

SEQ ID NO: 36 - DNA, 20 bp, Artificial Sequence
  primer
tccaccaccctgttgctgta

SEQ ID NO: 37 - DNA, 22 bp, Artificial Sequence
  primer
agaagcttctccctgacatccg

SEQ ID NO: 38 - DNA, 26 bp, Artificial Sequence
  primer
ggtaccaggagtgtttctccagactg

SEQ ID NO: 39 - DNA, 18 bp, Artificial Sequence
  primer
tgaaactccagactgtcc

SEQ ID NO: 40 - DNA, 20 bp, Artificial Sequence
  primer
tcagaccctgaggctcaaag

SEQ ID NO: 41 - DNA, 19 bp, Artificial Sequence
  primer
ccaaccgcgagaagatgac

SEQ ID NO: 42 - DNA, 20 bp, Artificial Sequence
  primer
tagcacagcctggatagcaa

SEQ ID NO: 43 - DNA, 20 bp, Artificial Sequence
  primer
cgcgagaagatgacccagat

SEQ ID NO: 44 - DNA, 20 bp, Artificial Sequence
  primer
cacagcctggatagcaacgt

SEQ ID NO: 45 - RNA, 57 nt, Artificial Sequence
  shRNA1
ccggggacaagcuauaucagaaacucucgagaguuucugauauagcuuguccuuuuu

SEQ ID NO: 46 - RNA, 57 nt, Artificial Sequence
  shRNA2
ccgggggccuggagauuuggaauagcucgagcuauuccaaaucuccaggcccuuuuu

SEQ ID NO: 47 - RNA, 57 nt, Artificial Sequence
  shRNA3
ccggggaauagucaggacagguucccucgagggaaccuguccugacuauuccuuuuu

SEQ ID NO: 48 - RNA, 57 nt, Artificial Sequence
  non-target_shRNA
ccgggcgcgauagcgcuaauaauuucucgagaaauuauuagcgcuaucgcgcuuuuu

SEQ ID NO: 49 - RNA, 21 nt, Homo sapiens
ggacaagcuauaucagaaacu

SEQ ID NO: 50 - RNA, 21 nt, Homo sapiens
gggccuggagauuuggaauag

SEQ ID NO: 51 - RNA, 21 nt, Homo sapiens
ggaauagucaggacagguucc

SEQ ID NO: 52 - RNA, 21 nt, Homo sapiens
gcgcgauagcgcuaauaauuu